(19) United States
(12) Patent Application Publication
(10) Pub. No.: US 2016/0120797 A1
(43) Pub. Date: May 5, 2016

(54) ANTI-ACNE COMPOSITION AND METHODS OF USE THEREOF

(71) Applicant: Sreedhar Rao Rayudu, Germantown, IN (US)

(72) Inventor: Sreedhar Rao Rayudu, Germantown, IN (US)

(21) Appl. No.: 14/927,334

(22) Filed: Oct. 29, 2015

Related U.S. Application Data
(60) Provisional application No. 62/073,770, filed on Oct. 31, 2014.

(51) Int. Cl. A61K 9/00; A61K 31/07; A61K 45/06; A61K 31/327; A61K 31/196; A61K 31/7056

(52) U.S. Cl. CPC A61K 9/0014; A61K 31/196; A61K 31/7056; A61K 45/06; A61K 31/327; A61K 31/07

(57) ABSTRACT

The present invention provides for a topical dermatological composition that includes diclofenac and clindamycin, in a pharmaceutically acceptable medium, wherein at least one of the diclofenac and clindamycin is at least partially microencapsulated.

### RELATED APPLICATIONS

This application claims priority to U.S. Ser. No. 62/073,770, filed Oct. 31, 2014, the contents of which are incorporated by reference herein in its entirety.

### BACKGROUND OF THE INVENTION

The burden of acne is significant. More than 50 million Americans experience some form of acne. Acne vulgaris is a common skin disorder that makes up 20% of the visits to dermatologists, and affects approximately 80% of young adults and adolescents. Management of acne is challenging, especially considering the chronicity of the disease and the variability in response to treatments. Acne management can be complex, because the disease is multifactorial, involving various etiological features, including follicular hyperkeratinisation, increased sebum production, P. acnes proliferation, and inflammation.

If not appropriately treated, acne may cause serious physical and emotional scarring and can significantly impact the quality of life of those affected by the disease.

Currently, acne treatment regimens have a wide variety of side effects and tolerability profiles, as well as differing efficacy in treatment. The need therefore exists to find alternative treatments for acne that improve the patient treatment experience.

### SUMMARY OF THE INVENTION

The present invention provides for a topical dermatological composition including a pharmaceutically acceptable medium and at least one nonsteroidal anti-inflammatory drug (NSAID) in the form of a microencapsulation, liposome, niosome, microsponge, microemulsion, micronized particles, microsphere, SLN, hydrogel, aerosol, or fullerene.

The present invention also provides a topical dermatological composition including:

(i) a pharmaceutically acceptable medium;

(ii) a nonsteroidal anti-inflammatory drug (NSAID);

(iii) optionally an antibiotic;

(iv) optionally an antibacterial agent;

(v) optionally a retinoid;

(vi) optionally an anti-androgenic agent;

(vii) optionally a folate;

(viii) optionally a keratolytic agent;

(ix) optionally at least one of azelaic acid, alpha hydroxy acid, trichloroacetic acid, lactic acid, ethanol, resorcinol, sulfur, sodium sulfacetamide, ivermectin, and glycolic acid; and

(x) optionally at least one penetrating agent;

wherein at least one of (a) and (b) is satisfied:

(a) any one or more of the components (ii)-(x) is microencapsulated, and

(b) at least one (e.g., 1, 2, 3, 4, 5, 6, 7, 8, 9, etc.) of (iii)-(ix) is present.

The present invention also provides for a topical dermatological composition including:

(i) a pharmaceutically acceptable medium;

(ii) a nonsteroidal anti-inflammatory drug (NSAID);

(iii) optionally an antibiotic;

(iv) optionally an antibacterial agent;

(v) optionally a retinoid;

(vi) optionally an anti-androgenic agent;

(vii) optionally a folate;

(viii) optionally a keratolytic agent;

(ix) optionally at least one of azelaic acid, alpha hydroxy acid, trichloroacetic acid, lactic acid, ethanol, resorcinol, sulfur, sodium sulfacetamide, ivermectin, and glycolic acid; and

(x) optionally at least one penetrating agent;

wherein any one or more of the components (ii)-(x) is optionally microencapsulated. In specific embodiments, at least one (e.g., 1, 2, 3, 4, 5, 6, 7, 8, 9, etc.) of (iii)-(ix) is present.

The present invention also provides for a topical dermatological composition including:

(i) a pharmaceutically acceptable medium;

(ii) a nonsteroidal anti-inflammatory drug (NSAID);

(iii) optionally an antibiotic;

(iv) optionally an antibacterial agent;

(v) optionally a retinoid;

(vi) optionally an anti-androgenic agent;

(vii) optionally a folate;

(viii) optionally a keratolytic agent;

(ix) optionally at least one of azelaic acid, alpha hydroxy acid, trichloroacetic acid, lactic acid, ethanol, resorcinol, sulfur, sodium sulfacetamide, ivermectin, and glycolic acid; and

(x) optionally at least one penetrating agent;

wherein at least one (e.g., 1, 2, 3, 4, 5, 6, 7, 8, 9, etc.) of (iii)-(ix) is present. In specific embodiments, any one or more of the components (ii)-(ix) is optionally microencapsulated.

In specific embodiments, the present invention provides for a topical dermatological composition that is long acting. In additional specific embodiments, the present invention provides for a topical dermatological composition that is suitable for the treatment of topical skin disorders such as acne. In additional specific embodiments, the present invention provides for a long acting topical dermatological formulation of NSAID medications, for the treatment of topical skin disorders such as acne.

The present invention also provides for a method of treating a topical skin disorder in a human patient. The method includes topically administering to a human patient in need thereof, the topical dermatological composition described herein, in an amount and for a period of time effective to treat the topical skin disorder.

The present invention also provides for a method of treating acne vulgaris in a human patient. The method includes topically administering to a human patient in need thereof, the topical dermatological composition described herein, in an amount and for a period of time effective to treat the acne vulgaris.

### DETAILED DESCRIPTION OF THE INVENTION

We believe that one skilled in the art can, based upon the description herein, use the present invention to its fullest extent. The following specific embodiments are to be construed as merely illustrative, and do not serve to limit the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

In reference to the compounds present in the topical dermatological compositions described herein, all pharmaceutically acceptable salts, amorphous form, crystalline forms, and solvates are intended.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

### DEFINITIONS

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

As used herein, the term “treating” or “treatment” refers to obtaining desired pharmacological and/or physiological effect. The effect can be prophylactic or therapeutic, which includes achieving, partially or substantially, one or more of the following results: partially or totally reducing the extent of the disease, disorder or syndrome; ameliorating or improving a clinical symptom or indicator associated with the disorder; delaying, inhibiting or decreasing the likelihood of the progression of the disease, disorder or syndrome; or partially or totally delaying, inhibiting or reducing the likelihood of the onset or development of disease, disorder or syndrome.

The term “topical” and its derivatives as used herein refers to directly laying on or spreading on the skin in need of the treatment, e.g., by use of the hands or an applicator.

The term “therapeutically effective amount” of a therapeutic agent as used herein refers to an amount of each active component of the pharmaceutical formulation that is sufficient to show a meaningful patient benefit, i.e., to cause a decrease in, amelioration of, or prevention of the symptoms of the condition being treated. Effective amounts of the pharmaceutical formulation will vary according to factors such as the degree of susceptibility of the individual, the age, gender, and weight of the individual and idiosyncratic responses of the individual.

The term “subject” as used herein refers to mammalian animals, preferably human.

As used herein, the term “fixed-dose” of the therapeutic agents refers to a combination dose that is administered to a human patient without regard for the weight (WT) or body surface area (BSA) of the patient. The fixed dose is therefore not provided as a mg/kg dose or a mg/m2 dose, but rather as an absolute amount of the therapeutic agent.

As used herein, “pharmaceutically-acceptable” means active agents, inert ingredients, or composition that are suitable for topical or oral administration without undue toxicity, incompatibility, instability, irritation, allergic response, and the like, commensurate with a reasonable benefit/risk ratio.

All active pharmaceutical ingredients (APIs) referenced herein can be present as the free compound, or as the pharmaceutically acceptable salt thereof.

The phrase “pharmaceutically acceptable salts” refers to ionic compounds wherein a parent non-ionic compound is modified by making acid or base salts thereof. For a review on pharmaceutically acceptable salts see Berge et al., J. Pharm. Sci. 1977, 66(1), 1-19, which is incorporated herein by reference. The pharmaceutically acceptable salts of the compounds described herein can be synthesized from the parent compound, which contains a basic or acidic moiety, by conventional chemical methods. Lists of many suitable salts are found in Remington's Pharmaceutical Sciences, 17th ed., Mack Publishing Company, Easton, Pa., (1985), 1418, and the disclosure of which is incorporated herein by reference.

As used herein, the term “topical dermatological composition” refers to a composition suitable for administration to an external body surface, such as the skin.

As used herein, the term “pharmaceutically acceptable medium” refers to a carrier or vehicle of one or more pharmaceutical medicaments that is safe and effective in the administration to a human patient, of the delivery of the one or more pharmaceutical medicaments.

As used herein, the term “nonsteroidal anti-inflammatory drug (NSAID)” refers to a class of drugs that provides analgesic (pain-killing) and antipyretic (fever-reducing) effects, and anti-inflammatory effects. The term “nonsteroidal” distinguishes these drugs from steroids, which, among a broad range of other effects, have a similar eicosanoid-depressing, anti-inflammatory action. As analgesics, NSAIDs are unusual in that they are non-narcotic and thus are used as a non-addictive alternative to narcotics. The most prominent members of this group of drugs (aspirin, ibuprofen and naproxen), are all available over the counter in most countries. NSAIDs inhibit the activity of both cyclooxygenase-1 (COX-1) and cyclooxygenase-2 (COX-2), and thereby, the synthesis of prostaglandins and thromboxanes.

As used herein, the term “microencapsulation” refers to a product obtained by the process in which tiny particles or droplets are surrounded by a coating to give small capsules of many useful properties. Microencapsulation can be used to enclose solids, liquids, or gases inside a micrometric wall made of hard or soft soluble film, in order to reduce dosing frequency and prevent the degradation of pharmaceuticals. As such, microencapsulation includes liposomes. In a relatively simple form, a microcapsule is a small sphere with a uniform wall around it. The material inside the microcapsule is referred to as the core, internal phase, or fill, whereas the wall is sometimes called a shell, coating, or membrane. Some materials like lipids and polymers, such as alginate, may be used as a mixture to trap the material of interest inside. Most microcapsules have pores with diameters between a few micrometers and a few millimeters. The coating materials typically used for coating are: (1) ethyl cellulose, (2) poly vinyl alcohol, (3) gelatin, and (4) sodium alginate.

As used herein, the term “liposome” is a specific type of microencapsulation, and refers to an artificially-prepared spherical vesicle composed of a lamellar phase lipid bilayer. The liposome can be used as a vehicle for administration of pharmaceutical drugs. Liposomes can be prepared by disrupting biological membranes (such as by sonication). Liposomes are often composed of phosphatidylcholine-enriched phospholipids and may also contain mixed lipid chains with surfactant properties such as egg phosphatidylethanolamine. A liposome design may employ surface ligands for attaching to unhealthy tissue. The major types of liposomes are the multilamellar vesicle (MLV), the small unilamellar liposome vesicle (SUV), the large unilamellar vesicle (LUV), and the cochleate vesicle. Liposomes should not be confused with micelles and reverse micelles composed of monolayers.

As used herein, the term “micronized particles” refers to a particle that has been micronized. Micronization is the process of reducing the average diameter of a solid material's particles. Usually, the term micronization is used when the particles that are produced are only a few micrometers in diameter. However, modern applications include average particle diameters of the nanometer scale.

As used herein, the term “microsphere” refers to are small spherical particles, with diameters in the micrometer range (typically 1 μm to 1000 μm (1 mm)) Microspheres can be manufactured from various natural and synthetic materials. Glass microspheres, polymer microspheres and ceramic microspheres are commercially available. Solid and hollow microspheres vary widely in density and, therefore, are used for different applications. Hollow microspheres are typically used as additives to lower the density of a material. Solid microspheres have numerous applications depending on what material they are constructed of and what size they are.

As used herein, the term “SLN” or “solid lipid nanoparticles” refers to a pharmaceutical delivery system that is typically spherical with—an average diameter between 10 to 1000 nanometers. Solid lipid nanoparticles possess a solid lipid core matrix that can solubilize lipophilic molecules. The lipid core is stabilized by surfactants (emulsifiers). The term lipid is used here in a broader sense and includes triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate). Due to their unique size-dependent properties, lipid nanoparticles offer the possibility to develop new therapeutics. The ability to incorporate drugs into nanocarriers offers a new prototype in drug delivery that could hold great promise for attaining the bioavailability enhancement along with controlled and site specific drug delivery. SLN's are also considered to be well tolerated in general, due to their composition from physiologically similar lipids. SLNs combine the advantages of lipid emulsion and polymeric nanoparticle systems while overcoming the temporal and in vivo stability issues that trouble the conventional as well as polymeric nanoparticles drug delivery approaches.

As used herein, the term “hydrogel” refers to a network of polymer chains that are hydrophilic, sometimes found as a colloidal gel in which water is the dispersion medium. Hydrogels are highly absorbent (they can contain over 90% water) natural or synthetic polymeric networks. Hydrogels also possess a degree of flexibility very similar to natural tissue, due to their significant water content. Many gels display thixotropy—they become fluid when agitated, but resolidify when resting. In general, gels are apparently solid, jelly-like materials. By replacing the liquid with gas it is possible to prepare aerogels, materials with exceptional properties including very low density, high specific surface areas, and excellent thermal insulation properties. Many substances can form gels when a suitable thickener or gelling agent is added to their formula. This approach is common in manufacture of wide range of products, from foods to paints and adhesives.

As used herein, the term “aerosol” refers to a type of dispensing system which creates an aerosol mist of liquid particles. This is used with a can or bottle that contains a liquid under pressure. When the container's valve is opened, the liquid is forced out of a small hole and emerges as an aerosol or mist. As gas expands to drive out the payload, only some propellant evaporates inside the can to maintain an even pressure. Outside the can, the droplets of propellant evaporate rapidly, leaving the payload suspended as very fine particles or droplets. Typical liquids dispensed in this way are insecticides, deodorants and paints. An atomizer is a similar device that is pressurized by a hand-operated pump rather than by stored gas.

As used herein, the term “fullerene” refers to any molecule composed of carbon in the form of a hollow sphere, ellipsoid, tube, and many other shapes. Spherical fullerenes are also called buckyballs, and they resemble the balls used in football (soccer). Cylindrical ones are called carbon nanotubes or buckytubes. Fullerenes are similar in structure to graphite, which is composed of stacked graphene sheets of linked hexagonal rings; but they may also contain pentagonal (or sometimes heptagonal) rings.

As used herein, the term “antibiotic” refers to an agent that either kills or inhibits the growth of a microorganism (e.g., virus, fungus, bacteria, protozoans, etc.). In specific embodiments, the antibiotic can include an oral antibiotic.

As used herein, the term “antibacterial agent” refers to an agent that either kills or inhibits the growth of bacteria.

As used herein, the term “retinoid” refers to a class of chemical compounds that are related chemically to vitamin A. There are three generations of retinoids: first generation retinoids: which include retinol, retinal, tretinoin (retinoic acid, Retin-A), isotretinoin, and alitretinoin; second generation retinoids: which include etretinate and its metabolite acitretin; and third generation retinoids: which include tazarotene, bexarotene and adapalene. In specific embodiments, the retinoid can include an oral retinoid.

As used herein, the term “anti-androgenic agent” or “androgen antagonist” refers to a compound that prevent androgens from expressing their biological effects on responsive tissues. Antiandrogens alter the androgen pathway by blocking the appropriate receptors, competing for binding sites on the cell's surface, or affecting androgen production. Antiandrogens are classified as steroidal or nonsteroidal. Antiandrogens inhibit circulating androgens by blocking androgen receptors, suppressing androgen synthesis, or acting in both those ways. The most common antiandrogens are androgen receptor (AR) antagonists which act on the target cell level and competitively bind to androgen receptors. In specific embodiments, the anti-androgenic agent can include an oral anti-androgenic agent.

As used herein, the term “folate” refers to the compound pteroyl-L-glutamic acid.

As used herein, the term “keratolytic agent” refers to a compound capable of removing the outer layer of the skin to loosen and shed; or softens keratin, a major component of the skin. Suitable compounds include, e.g., salicylic acid, sulphur, urea, lactic acid, and allantoin.

As used herein, the term “azelaic acid” or “nonanedioic acid” refers to a compound with the formula (CH₂)₇(CO₂H)₂.

As used herein, the term “alpha hydroxy acid” refers to a class of chemical compounds that consist of a carboxylic acid substituted with a hydroxyl group on the adjacent carbon. They may be either naturally occurring or synthetic. Many well-known α-hydroxy acids are useful building blocks in organic synthesis: the most common and simple are glycolic acid, lactic acid, citric acid, mandelic acid.

As used herein, the term “trichloroacetic acid” refers to is an analogue of acetic acid in which the three hydrogen atoms of the methyl group have all been replaced by chlorine atoms.

As used herein, the term “lactic acid” refers to the compound 2-hydroxypropanoic acid.

As used herein, the term “ethanol” or “ethyl alcohol” refers to the compound CH₃CH₂OH.

As used herein, the term “resorcinol” refers to the compound benzene-1,3-diol.

As used herein, the term “sulfur” refers to a chemical element with symbol S and atomic number 16. Elemental sulfur is a bright yellow crystalline solid when at room temperature. Elemental sulfur oxidizes slowly to sulfurous acid, which in turn (through the action of sulfite) acts as a mild reducing and antibacterial agent.

As used herein, the term “sodium sulfacetamide” refers to the sodium salt of N-[(4-aminophenyl)sulfonyl]acetamide.

As used herein, the term “ivermectin” refers to the combination of compounds 22,23-dihydro avermectin B1a+22,23-dihydroavermectin B1b.

As used herein, the term “glycolic acid” refers to the compound 2-hydroxyethanoic acid.

As used herein, the term “penetrating agent” refers to a substance that assists with one or more other compounds in penetrating the skin, thereby at least partially crossing the skin barrier.

As used herein, the term “cyclooxygenase (COX) inhibitor” refers to a compound that inhibits the COX enzyme. Cyclooxygenase (COX), officially known as prostaglandin-endoperoxide synthase (PTGS), is an enzyme that is responsible for formation of important biological mediators called prostanoids, including prostaglandins, prostacyclin and thromboxane. COX includes both COX-1 and COX-2. Pharmacological inhibition of COX can provide relief from the symptoms of inflammation and pain. Non-steroidal anti-inflammatory drugs (NSAID), such as aspirin and ibuprofen, exert their effects through inhibition of COX. The names “prostaglandin synthase (PHS)” and “prostaglandin endoperoxide synthetase (PES)” are still used to refer to COX. The main COX inhibitors are the non-steroidal anti-inflammatory drugs (NSAIDs).

As used herein, the term “flufenamic acid” refers to the compound 2-{[3-(trifluoromethyl)phenyl]amino}benzoic acid.

As used herein, the term “meclofenamic acid” refers to the compound 2-[(2,6-dichloro-3-methylphenyl)amino]benzoic acid.

As used herein, the term “mefenamic acid” refers to the compound 2-(2,3-dimethylphenyl)aminobenzoic acid.

As used herein, the term “tolfenamic acid” refers to the compound 2-[(3-chloro-2-methylphenyl)amino]benzoic acid).

As used herein, the term “alminoprofen” refers to the compound 2-[4-(2-methylprop-2-enylamino)phenyl]propanoic acid.

As used herein, the term “carprofen” refers to the compound (RS)-2-(6-Chloro-9H-carbazol-2-yl)propanoic acid.

As used herein, the term “dexibuprofen” refers to the compound (2S)-2-[4-(2-methylpropyl)phenyl]propanoic acid.

As used herein, the term “dexketoprofen” refers to the compound (2S)-2-[3-(benzoyl)phenyl]propanoic acid.

As used herein, the term “fenbufen” refers to the compound 4-(4-biphenylyl)-4-oxobutanoic acid.

As used herein, the term “fenoprofen” refers to the compound 2-(3-phenoxyphenyl)propanoic acid.

As used herein, the term “flunoxaprofen” refers to the compound 2S)-2-[2-(4-fluorophenyl)-1,3-benzoxazol-5-yl]propanoic acid.

As used herein, the term “flurbiprofen” refers to the compound (RS)-2-(2-fluorobiphenyl-4-yl)propanoic acid.

As used herein, the term “ibuprofen” refers to the compound (RS)-2-(4-(2-methylpropyl)phenyl)propanoic acid.

As used herein, the term “ibuproxam” refers to the compound N-hydroxy-2-(4-isobutylphenyl)propanamide.

As used herein, the term “indoprofen” refers to the compound 2-[4-(1-oxo-1,3-dihydro-2H-isoindol-2-yl)phenyl]propanoic acid.

As used herein, the term “ketoprofen” refers to the compound (RS)-2-(3-benzoylphenyl)propanoic acid.

As used herein, the term “ketorolac” refers to the compound (±)-5-benzoyl-2,3-dihydro-1H-pyrrolizine-1-carboxylic acid, 2-amino-2-(hydroxymethyl)-1,3-propanediol.

As used herein, the term “loxoprofen” refers to the compound (RS)-2-{4-[(2-oxocyclopentyl)methyl]phenyl}propanoic acid.

As used herein, the term “naproxen” refers to the compound (+)-(S)-2-(6-methoxynaphthalen-2-yl)propanoic acid.

As used herein, the term “oxaprozin” refers to the compound 3-(4,5-diphenyl-1,3-oxazol-2-yl)propanoic acid.

As used herein, the term “pirprofen” refers to the compound 2-[3-chloro-4-(2,5-dihydro-1H-pyrrol-1-yl)phenyl]propanoic acid.

As used herein, the term “suprofen” refers to the compound (RS)-2-[4-(2-thienylcarbonyl)phenyl]propanoic acid.

As used herein, the term “tiaprofenic acid” refers to the compound (RS)-2-(5-benzoyl-2-thienyl)propanoic acid.

As used herein, the term “aceclofenac” refers to the compound 2-[2-[2-[(2,6-dichlorophenyl)amino]phenyl]acetyl]oxyacetic acid.

As used herein, the term “acemetacin” refers to the compound 2-[2-[1-(4-chlorobenzoyl)-5-methoxy-2-methylindol-3-yl]acetyl]oxyacetic acid.

As used herein, the term “alclofenac” refers to the compound 2-(3-chloro-4-prop-2-enoxyphenyl)acetic acid.

As used herein, the term “bromfenac” refers to the compound 2-[2-amino-3-(4-bromobenzoyl)phenyl]acetic acid.

As used herein, the term “diclofenac” refers to the compound 2-(2-(2,6-dichlorophenylamino)phenyl)acetic acid. In specific embodiments, the diclofenac can be diclofenac sodium.

As used herein, the term “etodolac” refers to the compound (RS)-2-(1,8-diethyl-4,9-dihydro-3H-pyrano[3,4-b]indol-1-yl)acetic acid.

As used herein, the term “indomethacin” refers to the compound 2-{1-[(4-chlorophenyl)carbonyl]-5-methoxy-2-methyl-1H-indol-3-yl}acetic acid.

As used herein, the term “nabumetone” refers to the compound 4-(6-methoxy-2-naphthyl)-2-butanone.

As used herein, the term “oxametacin” refers to the compound 2-[1-(4-chlorobenzoyl)-5-methoxy-2-methyl-1H-indol-3-yl]-N-hydroxyacetamide.

As used herein, the term “proglumetacin” refers to the compound 3-{4-[2-({[1-(4-chlorobenzoyl)-5-methoxy-2-methyl-1H-indol-3-yl]acetyl}oxy)ethyl]piperazin-1-yl}propyl N2-benzoyl-N,N-dipropyl-α-glutaminate.

As used herein, the term “sulindac” refers to the compound {(1Z)-5-fluoro-2-methyl-1-[4-(methylsulfinyl)benzylidene]-1H-indene-3-yl}acetic acid.

As used herein, the term “tolmetin” refers to the compound [1-methyl-5-(4-methylbenzoyl)-1H-pyrrol-2-yl]acetic acid.

As used herein, the term “erythromycin” refers to the compound (3R,4S,5S,6R,7R,9R,11R,12R,13S,14R)-6-{[(2S,3R,4S,6R)-4-(dimethylamino)-3-hydroxy-6-methyloxan-2-yl]oxy}-14-ethyl-7,12,13-trihydroxy-4-{R2R,4R,5S,6S)-5-hydroxy-4-methoxy-4,6-dimethyloxan-2-yl}oxyl-3,5,7,9,11,13-hexamethyl-1-oxacyclotetradecane-2,10-dione.

As used herein, the term “metronidazole” refers to the compound 2-(2-methyl-5-nitro-1H-imidazol-1-yl)ethanol

As used herein, the term “clindamycin” refers to the compound methyl 7-chloro-6,7,8-trideoxy-6-{[(4R)-1-methyl-4-propyl-L-prolyl]amino}-1-thio-L-threo-α-D-galacto-octopyranoside. In specific embodiments, the clindamycin can be clindamycin phosphate.

As used herein, the term “doxycycline” refers to the compound (4S,4aR,5S,5aR,6R,12aS)-4-(dimethylamino)-3,5,10,12,12a-pentahydroxy-6-methyl-1,11-dioxo-1,4,4a,5,5a,6,11,12a-octahydrotetracene-2-carboxamide (4S,4aR,5S,5aR,6R,12aS)-4-(dimethylamino)-3,5,10,12,12a-pentahydroxy-6-methyl-1,11-dioxo-1,4,4a,5,5a,6,11,12a-octahydrotetracene-2-carboxamide.

As used herein, the term “minocycline” refers to the compound (2E,4S,4aR,5aS,12aR)-2-(amino-hydroxy-methylidene)-4,7-bis(dimethylamino)-10,11,12a-trihydroxy-4a,5,5a,6-tetrahydro-4H-tetracene-1,3,12-trione.

As used herein, the term “tetracycline” refers to the compound (4S,6S,12aS)-4-(dimethylamino)-1,4,4a,5,5a,6,11,12a-octahydro-3,6,10,12,12a-pentahydroxy-6-methyl-1,11-dioxonaphthacene-2-carboxamide.

As used herein, the term “oxytetracycline” refers to the compound (4S,4aR,5S,5aR,6S,12aS)-4-(dimethylamino)-3,5,6,10,11,12a-hexahydroxy-6-methyl-1,12-dioxo-1,4,4a,5,5a,6,12,12a-octahydrotetracene-2-carboxamide.

As used herein, the term “chlortetracycline” refers to the compound (4S,4aS,5aS,6S,12aS,Z)-2-[amino(hydroxy)methylene]-7-chloro-4-(dimethylamino)-6,10,11,12a-tetrahydroxy-6-methyl-4a,5,5a,6-tetrahydrotetracene-1,3,12(2H,4H,12aH)-trione.

As used herein, the term “dapsone” refers to the compound 4-[(4-aminobenzene)sulfonyl]aniline.

As used herein, the term “demeclocycline” refers to the compound (2E,4S,4aS,5aS,6S,12aS)-2-[amino(hydroxy)methylidene]-7-chloro-4-(dimethylamino)-6,10,11,12a-tetrahydroxy-1,2,3,4,4a,5,5a,6,12,12a-decahydrotetracene-1,3,12-trione.

As used herein, the term “retinol” refers to the compound (2E,4E,6E,8E)-3,7-dimethyl-9-(2,6,6-trimethylcyclohex-1-enyl)nona-2,4,6,8-tetraen-1-ol.

As used herein, the term “retinal” refers to the compound (2E,4E,6E,8E)-3,7-Dimethyl-9-(2,6,6-trimethylcyclohexen-1-yl)nona-2,4,6,8-tetraenal.

As used herein, the term “tretinoin” or “Retin-A” refers to the compound retinoic acid.

As used herein, the term “isotretinoin” refers to the compound (2Z,4E,6E,8E)-3,7-dimethyl-9-(2,6,6-trimethylcyclohex-1-en-1-yl)nona-2,4,6,8-tetraenoic acid.

As used herein, the term “alitretinoin” refers to the compound (2E,4E,6Z,8E)-3,7-dimethyl-9-(2,6,6-trimethyl-1-cyclohexenyl)nona-2,4,6,8-tetraenoic acid.

As used herein, the term “etretinate” refers to the compound ethyl 9-(4-methoxy-2,3,6-trimethyl-phenyl)-3,7-dimethyl-nona-2,4,6,8-tetraenoate.

As used herein, the term “acitretin” refers to the compound (2E,4E,6E,8E)-9-(4-methoxy-2,3,6-trimethylphenyl)-3,7-dimethylnona-2,4,6,8-tetraenoic acid.

As used herein, the term “tazarotene” refers to the compound ethyl 6-[2-(4,4-dimethyl-3,4-dihydro-2H-1-benzothiopyran-6-yl)ethynyl]pyridine-3-carboxylate.

As used herein, the term “bexarotene” refers to the compound 4-[1-(3,5,5,8,8-pentamethyltetralin-2-yl)ethenyl]benzoic acid.

As used herein, the term “adapalene” refers to the compound 6-[3-(1-adamantyl)-4-methoxy-phenyl]naphthalene-2-carboxylic acid.

As used herein, the term “ASC-J9” refers to the compound (1E,4Z,6E)-1,7-bis(3,4-dimethoxyphenyl)-5-hydroxyhepta-1,4,6-trien-3-one.

As used herein, the term “antihistamine” or “histamine antagonist” refers to a pharmaceutical drug that inhibits the action of histamine by either blocking its attachment to histamine receptors, or inhibiting the enzymatic activity of histidine decarboxylase which catalyzes the transformation of histidine into histamine (atypical antihistaminics).

As used herein, the term “salicylic acid” refers to the compound 2-hydroxybenzoic acid.

As used herein, the term “benzoyl peroxide” refers to the compound dibenzoyl peroxide.

As used herein, the term “co-trimoxazole” refers to the combination of trimethoprim (the compound designated as 5-(3,4,5-trimethoxybenzyl)pyrimidine-2,4-diamine) and sulfamethoxazole (the compound designated as 4-amino-N-(5-methylisoxazol-3-yl)-benzenesulfonamide).

As used herein, “tea tree oil” refers to an essential oil with a fresh camphoraceous odor and a color that ranges from pale yellow to nearly colorless and clear. It is taken from the leaves of the Melaleuca alternifolia, which is native to Southeast Queensland and the Northeast coast of New South Wales, Australia.

As used herein, “healing oils” refers to any one or more essential oils considered to be effective in a variety of dermatologic conditions. Suitable substances useful in the healing oils include, e.g., green tea extract, jojoba oil, argan oil, turmeric, and capsaicin.

As used herein, “almond extract” refers to the extract (e.g., oil) of the edible and widely cultivated seed from the tree Prunus dulcis.

As used herein, “lauric acid” refers to the compound dodecanoic acid.

As used herein, “cyproterone acetate” refers to the compound (1R,3aS,3bR,7aR,8aS,8bS,8cS,10aS)-1-acetyl-5-chloro-8b,10a-dimethyl-7-oxo-1,2,3,3a,3b,7,7a,8,8a,8b,8c,9,10,10a-tetradecahydrocyclopenta[a]cyclopropa[g]phenanthren-1-yl acetate.

As used herein, the term “NSAID” or “nonsteroidal anti-inflammatory drugs” refers to a class of drugs that provides analgesic (pain-killing) and antipyretic (fever-reducing) effects, and anti-inflammatory effects. The term nonsteroidal distinguishes these drugs from steroids, which, among a broad range of other effects, have a similar eicosanoid-depressing, anti-inflammatory action. As analgesics, NSAIDs are unusual in that they are non-narcotic and thus are used as a non-addictive alternative to narcotics.

As used herein, the term “cleanser or wash” refers to a skin care product that is used to remove make-up, dead skin cells, oil, dirt, and other types of pollutants from the skin of the body and face. This helps to unclog pores and prevent skin conditions such as acne.

As used herein, the term “pledget” or “pledget wipe” refers to a medication that is applied to body surfaces such as the skin. The pledget includes a topical solution on a topical applicator. The topical applicator is typically a woven or non-woven fabric. As the topical applicator contacts the body surface, the solution is applied to the skin. The pledglets are typically single-use items, configured for disposal after use.

As used herein, the term “solution” or “topical solution” refers to a medication that is applied to body surfaces such as the skin. A solution is a homogeneous mixture composed of only one phase. In such a mixture, a solute is a substance dissolved in another substance, known as a solvent. The solution more or less takes on the characteristics of the solvent including its phase, and the solvent is commonly the major fraction of the mixture. The concentration of a solute in a solution is a measure of how much of that solute is dissolved in the solvent.

As used herein, the term “suspension” or “topical suspension” refers to a medication that is applied to body surfaces such as the skin. A suspension is a heterogeneous mixture containing solid particles that are sufficiently large for sedimentation. Usually they must be larger than one micrometer. The internal phase (solid) is dispersed throughout the external phase (fluid) through mechanical agitation, with the use of certain excipients or suspending agents.

Unlike colloids, suspensions will eventually settle. An example of a suspension would be sand in water. The suspended particles are visible under a microscope and will settle over time if left undisturbed. This distinguishes a suspension from a colloid, in which the suspended particles are smaller and do not settle. Colloids and suspensions are different from solutions, in which the dissolved substance (solute) does not exist as a solid, and solvent and solute are homogeneously mixed.

As used herein, the term “foam” or “topical foam” refers a medication that is applied to body surfaces such as the skin. A foam is a substance that is formed by trapping pockets of gas in a liquid or solid. In most foams, the volume of gas is large, with thin films of liquid or solid separating the regions of gas.

As used herein, the term “cream” or “topical cream” refers to a medication that is applied to body surfaces such as the skin. Creams are semi-solid emulsions that are mixtures of oil and water. They are divided into two types: oil-in-water (O/W) creams which are composed of small droplets of oil dispersed in a continuous phase, and water-in-oil (W/O) creams which are composed of small droplets of water dispersed in a continuous oily phase. Oil-in-water creams are more comfortable and cosmetically acceptable as they are less greasy and more easily washed off using water. Water-in-oil creams are more difficult to handle but many drugs which are incorporated into creams are hydrophobic and will be released more readily from a water-in-oil cream than an oil-in-water cream. Water-in-oil creams are also more moisturizing as they provide an oily barrier which reduces water loss from the stratum corneum, the outermost layer of the skin. Creams are viscous liquids or semi-solid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

As used herein, the terms “aqueous gel” and “non-aqueous gel” refer to a medication that is applied to body surfaces such as the skin. A gel is a solid, jelly-like material that can have properties ranging from soft and weak to hard and tough. Gels are defined as a substantially dilute cross-linked system, which exhibits no flow when in the steady-state. By weight, gels are mostly liquid, yet they behave like solids due to a three-dimensional cross-linked network within the liquid. It is the crosslinking within the fluid, that give a gel its structure (hardness) and contribute to the adhesive stick (tack). In this way gels are a dispersion of molecules of a liquid within a solid in which the solid is the continuous phase and the liquid is the discontinuous phase. When the liquid includes water, the gel is considered an aqueous gel. In contrast, when the liquid does not include water, the gel is considered a non-aqueous gel.

As used herein, the term “ointment” refers to a medication that is applied to body surfaces such as the skin. Ointments are homogeneous, viscous, semi-solid preparation, most commonly greasy, thick oil (oil 80%—water 20%) with a high viscosity, intended for external application to the skin or mucous membranes. Ointments have a Water number that defines the maximum amount of water that it can contain. They are used as emollients or for the application of active ingredients to the skin for protective, therapeutic, or prophylactic purposes and where a degree of occlusion is desired. Ointments are semi-solid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable-bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols of varying molecular weight; again, see Remington: The Science and Practice of Pharmacy for further information.

As used herein, the term “emulsion” refers to a medication that is applied to body surfaces such as the skin. An emulsion is a mixture of two or more liquids that are normally immiscible (nonmixable or unblendable). Emulsions are part of a more general class of two-phase systems of matter called colloids.

Although the terms colloid and emulsion are sometimes used interchangeably, emulsion should be used when both the dispersed and the continuous phase are liquids. In an emulsion, one liquid (the dispersed phase) is dispersed in the other (the continuous phase).

As used herein, the term “oil” refers to a medication that is applied to body surfaces such as the skin. Oils are neutral, nonpolar chemical substances that are viscous liquid at ambient temperatures and both hydrophobic (immiscible with water, literally “water fearing”) and lipophilic (miscible with other oils, literally “fat loving”). Oils have a high carbon and hydrogen content and are usually flammable and slippery.

As used herein, the term “toner” or “skin toner” refers to a medication that is applied to body surfaces such as the skin. A toner is a lotion or wash designed to cleanse the skin and shrink the appearance of pores, usually used on the face.

As used herein, the term “lotion” refers to a medication that is applied to body surfaces such as the skin. A lotion is a low- to heavy-viscosity topical preparation intended for application to unbroken skin. By contrast, creams and gels have higher viscosity. Lotions are typically semi-liquid preparations in which solid particles, including the active agent, are present in a water or alcohol base. Lotions are usually suspensions of solids, and preferably, for the present purpose, include a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

As used herein, the term “aerosol” or “aerosol spray” refers to a medication that is applied to body surfaces such as the skin. An aerosol spray is a colloid of fine solid particles or liquid droplets, in air or another gas. Aerosol spray is a type of dispensing system which creates an aerosol mist of liquid particles. This is used with a can or bottle that contains a liquid under pressure. When the container's valve is opened, the liquid is forced out of a small hole and emerges as an aerosol or mist. As gas expands to drive out the payload, only some propellant evaporates inside the can to maintain an even pressure. Outside the can, the droplets of propellant evaporate rapidly, leaving the payload suspended as very fine particles or droplets. Typical liquids dispensed in this way are insecticides, deodorants and paints. An atomizer is a similar device that is pressurized by a hand-operated pump rather than by stored gas.

As used herein, the term “topical adhesive patch” refers to a medication that is applied to body surfaces such as the skin. A topical adhesive patch is a medicated adhesive patch that is placed on the skin to deliver a medication into the skin. This is in contrast to a transdermal patch, which delivers the medication completely through the skin and into the bloodstream.

As used herein, the term “medicament” refers to is any chemical substance formulated or compounded as single active ingredient or in combination of other pharmacologically active substance, it may be in a separate but packed in a single unit pack as combination product intended for internal, or external or for use in the medical diagnosis, cure, treatment, or prevention of a disease or disorder.

As used herein, the term “fragrance” refers to a chemical compound that has a smell or odor. A chemical compound has a smell or odor when it is sufficiently volatile to be transported to the olfactory system in the upper part of the nose.

As used herein, the term “preserving agent” refers to a substance that is added to products such as pharmaceuticals to prevent decomposition by microbial growth or by undesirable chemical changes. In general preservation is implemented in two modes, chemical and physical. Chemical preservation entails adding chemical compounds to the product.

As used herein, the term “stabilizer” refers to a substance added to prevent unwanted change in state of another substance. A stabilizer is a chemical which tends to inhibit the reaction between two or more other chemicals. Some kinds of stabilizers include, e.g., antioxidants, sequestrants, emulsifiers, surfactants, and ultraviolet stabilizers.

As used herein, the term “pH regulator” refers to a substance capable of altering the acidity or basicity of an aqueous solution.

As used herein, the term “antioxidant” refers to a molecule that inhibits the oxidation of other molecules.

As used herein, the term “topical skin disorder” refers to a skin disorder affecting a topical skin surface (e.g., face, neck, shoulders, and/or upper back). One specific topical skin disorder is acne vulgaris.

As used herein, the term “human patient” refers to a patient that is human (i.e., extant members of the hominin clade, a branch of great apes characterized by erect posture and bipedal locomotion; manual dexterity and increased tool use; and a general trend toward larger, more complex brains and societies).

As used herein, the term “acne vulgaris” refers to a common human skin disease, characterized by any of the following: areas of seborrhea (scaly red skin), comedones (blackheads and whiteheads), papules (pinheads), nodules (large papules), pimples, and possibly scarring. Aside from scarring, its main effects are psychological, such as reduced self-esteem and in very extreme cases, depression or suicide.

As used herein, the term “chemical peel” refers to a body treatment technique used to improve and smooth the texture of the facial skin using a chemical solution that causes the dead skin to slough off and eventually peel off. The regenerated skin is usually smoother and less wrinkled than the old skin.

As used herein, the term “oral anti-androgen” refers to medications taken by mouth for the purpose of altering the androgen pathway by blocking the appropriate receptors, competing for binding sites on the cell's surface, or affecting androgen production.

As used herein, the term “spironolactone” refers to the compound 7a-acetylthio-3-oxo-17a-pregn-4-ene-21,17-carbolactone.

As used herein, the term “cimetidine” refers to the compound 1-cyano-2-methyl-3-[2-[(5-methyl-1H-imidazol-4-yl)methylsulfanyl]ethyl]guanidine.

As used herein, the term “oral contraceptive” refers to medications taken by mouth for the purpose of birth control, containing both synthetic estrogens and progestogens (progestins), and progestogen only pills (mini-pills).

As used herein, the term “photodynamic therapy” refers to the exposure to daylight or to specific wavelengths of light using polychromatic polarized light, lasers, light-emitting diodes, fluorescent lamps, dichroic lamps or very bright, full-spectrum light. The light is administered for a prescribed amount of time and, in some cases, at a specific time of day. Two forms of phototherapy exist: non-targeted phototherapy (from sunlight or a light box), and targeted phototherapy, in which light is administered to a specific, localized area of the skin. Current targeted phototherapy is administered via excimer laser, elemental gas lamp, or via LED light.

As used herein, the term “oral antibiotic” refers to medications taken by mouth for the purpose of either killing or inhibiting the growth of a microorganism.

As used herein, the term “doxycycline” refers to the compound (4S,4aR,5S,5aR,6R,12aS)-4-(dimethylamino)-3,5,10,12,12a-pentahydroxy-6-methyl-1,11-dioxo-1,4,4a,5,5a,6,11,12a-octahydrotetracene-2-carboxamide.

As used herein, the term “minocycline” refers to the compound (2E,4S,4aR,5aS,12aR)-2-(amino-hydroxy-methylidene)-4,7-bis(dimethylamino)-10,11,12a-trihydroxy-4a,5,5a,6-tetrahydro-4H-tetracene-1,3,12-trione.

As used herein, the term “adolescent” refers to a human patient between the ages of about 10 and 18. Such a person would be pre-pubescent, pubescent, or post-pubescent.

### Liposomes and NSAIDs

Liposomes are a preferred microencapsulation method for NSAID drugs and may include one or more of an antibiotic, antibacterial, or retinoid agent to achieve the combination therapy effective in acne. Controlled release of active drugs has several advantages, including reduced skin irritation and allowing drug with a short half-life to be effective over longer periods of time. Several known methods of formulating liposome types may provide adequate delivery of NSAID medications, as described herein.

Transferosomes are composed of phospholipid, optionally between 5-20% ethanol, 10-20% surfactant (Tween 80/Span 80), and may include cholesterol. Traditional liposome manufacturing methods may be employed, with dissolution of lipid in chloroform, rotary evaporation to remove organic solvent and create a thin lipid layer, and hydration with aqueous or ethanolic solutions containing NSAID followed by membrane extrusion.

Conventional liposomes can be composed of phospholipid bilayers alone or may include cholesterol and are prepared in similar fashion.

Other dermal delivery options include a 2-10 micron sized inert polymers assembled to form microspheres. At this size, they have been shown to target follicles, and so a diclofenac loaded microsphere may achieve controlled release in a fashion similar to liposomes. Alternatively, slow release gels may also act as a reservoir to allow diclofenac to permeate the skin over time.

The carriers useful for topical delivery of the specified topical dermatological compositions according to embodiments of the invention can be any carrier known in the art for topically administering pharmaceuticals, including, but not limited to, pharmaceutically acceptable solvents, such as a polyalcohol or water; emulsions (either oil-in-water or water-in-oil emulsions), such as creams or lotions; micro emulsions; gels; ointments; liposomes; powders; and aqueous solutions or suspensions. The pharmaceutically acceptable carrier includes necessary and inert pharmaceutical excipients, including, but not limited to, binders, suspending agents, lubricants, fragrances, preservatives, dyes, and coatings.

The topical composition according to embodiments of the present invention are prepared by mixing a pharmaceutically acceptable carrier with a therapeutically effective amount of active pharmaceutical ingredient(s) according to known methods in the art, for example, methods provided by standard reference texts such as, REMINGTON: THE SCIENCE AND PRACTICE OF PHARMACY 1577-1591, 1672-1673, 866-885 (Alfonso R. Gennaro ed. 1 9th ed. 1995); TRANSDERMAL AND TOPICAL DRUG DELIVERY SYSTEMS (Ghosh, T. K., et al. ed. 1997), both of which are hereby incorporated herein by reference.

In one embodiment, the topical composition of the invention is in the form of an emulsion. Emulsions, such as creams and lotions are suitable topical formulations for use in the invention. An emulsion is a dispersed system including at least two immiscible phases, one phase dispersed in the other as droplets ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout the aqueous phase, the emulsion is termed an oil-in-water emulsion. Emulsions, such as creams and lotions that can be used as topical carriers and their preparation are disclosed in REMINGTON: THE SCIENCE AND PRACTICE OF PHARMACY 282-291 (Alfonso R. Gennaro ed. 19th ed. 1995), hereby incorporated herein by reference.

In another embodiment, the topical composition of the invention is in the form of a gel, for example, a two-phase gel or a single-phase gel. Gels are semisolid systems consisting of suspensions of small inorganic particles or large organic molecules interpenetrated by a liquid. When the gel mass includes a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are disclosed in REMINGTON: THE SCIENCE AND PRACTICE OF PHARMACY 1517-1518 (Alfonso R. Gennaro ed. 19th ed. 1995), hereby incorporated herein by reference. Other suitable gels for use with the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002), each of which patents is hereby incorporated herein by reference.

In an embodiment, the topical composition further includes an aqueous gel including water and a water-gelling amount of a pharmaceutically acceptable gelling agent selected from carbomers, glycerine polyacrylate, and mixtures thereof, and the topical composition has a physiologically acceptable pH.

As used herein, “carbomer” is the USP designation for various polymeric acids that are dispersible but insoluble in water. When the acid dispersion is neutralized with a base a clear, stable gel is formed. Carbomer 934P is physiologically inert and is not a primary irritant or sensitizer. Other carbomers include 910, 940, 941, and 1342. Polymer thickeners (gelling agents) that may be used in compositions according to embodiments of the present invention include those known to one skilled in the art, such as hydrophilic and hydroalcoholic gelling agents frequently used in the cosmetic and pharmaceutical industries. Preferably, the hydrophilic or hydroalcoholic gelling agent includes “CARBOPOL®” (B.F. Goodrich, Cleveland, Ohio), “HYP AN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.). Preferably the gelling agent includes between about 0.2% to about 4% by weight of the composition. More particularly, the preferred compositional weight percent range for “CARBOPOL®” is between about 0.5% to about 2%, while the preferred weight percent range for “NATROLSOL®” and “KLUCEL®” is between about 0.5% to about 4%. The preferred compositional weight percent range for both “HYPAN®” and “STABILEZE®” is between 0.5% to about 4%.

“CARBOPOL®” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. “KLUCEL®” is a cellulose polymer that is dispersed in water and forms a uniform gel upon complete hydration. Other preferred gelling polymers include hydroxyethylcellulose, cellulose gum, MVE/MA decadiene crosspolymer, PVM/MA copolymer, or a combination thereof.

In another embodiment, the topical composition of the invention is in the form of an ointment. Ointments are oleaginous semisolids that contain little if any water.

Specifically, the ointment can be hydrocarbon based, such as a wax, petrolatum, or gelled mineral oil. Suitable ointments for use in the invention are well known in the art and are disclosed in REMINGTON: THE SCIENCE AND PRACTICE OF PHARMACY 1585-1591 (Alfonso R. Gennaro ed. 19th ed. 1995), hereby incorporated herein by reference.

In an embodiment of the present invention, the topical composition of the invention includes at least one of a cream and an ointment, each including an agent selected from stearic acid, stearyl alcohol, cetyl alcohol, glycerin, water, and mixtures thereof, and the topical composition has a physiologically acceptable pH.

In another embodiment, the topical composition of the invention can be in the form of an aqueous solution or suspension, specifically, an aqueous solution. Suitable aqueous topical formulations for use in the invention include those disclosed in REMINGTON: THE SCIENCE AND PRACTICE OF PHARMACY 1563-1576 (Alfonso R. Gennaro ed. 19th ed. 1995), hereby incorporated herein by reference. Other suitable aqueous topical carrier systems include those disclosed in U.S. Pat. No. 5,424,078 (issued Jun. 13, 1995); U.S. Pat. No. 5,736,165 (issued Apr. 7, 1998); U.S. Pat. No. 6,194,415 (issued Feb. 27, 2001); U.S. Pat. No. 6,248,741 (issued Jun. 19, 2001); and U.S. Pat. No. 6,465,464 (issued Oct. 15, 2002), all of which patents are hereby incorporated herein by reference.

The pH of the topical formulations of the invention are preferably within a physiologically acceptable pH, e.g., within the range of about 5 to about 8, more preferably, of about 5.5 to about 6.5. To stabilize the pH, preferably, an effective amount of a buffer is included. In one embodiment, the buffering agent is present in the aqueous topical formulation in an amount of from about 0.05 to about 1 weight percent of the formulation. Acids or bases can be used to adjust the pH as needed.

Tonicity-adjusting agents can be included in the aqueous topical formulations of the invention. Examples of suitable tonicity-adjusting agents include, but are not limited to, sodium chloride, potassium chloride, mannitol, dextrose, glycerin, and propylene glycol. The amount of the tonicity agent can vary widely depending on the formulation's desired properties. In one embodiment, the tonicity-adjusting agent is present in the aqueous topical formulation in an amount of from about 0.5 to about 0.9 weight percent of the formulation.

Specifically, the aqueous topical formulations of the invention can have a viscosity in the range of from about 15 cps to about 25 cps. The viscosity of aqueous solutions of the invention can be adjusted by adding viscosity adjusting agents, for example, but not limited to, polyvinyl alcohol, povidone, hydroxypropyl methyl cellulose, poloxamers, carboxymethyl cellulose, or hydroxyethyl cellulose.

In a specific embodiment, the aqueous topical formulation of the invention can be isotonic saline including a preservative, such as benzalkonium chloride or chlorine dioxide, a viscosity-adjusting agent, such as polyvinyl alcohol, and a buffer system such as sodium citrate and citric acid.

The topical composition according to embodiments of the invention can include pharmaceutically acceptable excipients such as those listed in REMINGTON: THE SCIENCE AND PRACTICE OF PHARMACY 866-885(Alfonso R. Gennaro ed. 19th ed. 1995); and TRANSDERMAL AND TOPICAL DRUG DELIVERY SYSTEMS (Ghosh, T. K. et al. ed. 1997), hereby incorporated herein by reference, including, but not limited to, protectives, adsorbents, demulcents, emollients, preservatives, antioxidants, moisturizers, buffering agents, solubilizing agents, skin-penetration agents, and surfactants.

In an embodiment, the topical composition of the invention further includes one or more agents selected from a preservative, a local anesthetic and a skin humectant.

Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenyl ethyl alcohol, and benzyl alcohol; antibacterial esters, for example, esters of parahydroxybenzoic acid; and other anti-microbial agents such as chlorhexidine, chlorocresol, benzoic acid and polymyxin.

In a specific embodiment, a topical composition according to embodiments of the invention further includes titanium dioxide (TiO2), preferably at an amount that is sufficient to mask the color of a colored ingredient in the formulation, but would not cause irritation to the skin. TiO2 may cause mild irritation and reddening to the eyes, thus eye contact with the TiO2-containing topically administrable composition should be avoided.

Dosages and dosing frequency will be determined by a trained medical professional depending on the activity of the compounds used, the characteristics of the particular topical formulation, and the identity and severity of the dermatologic disorder treated or prevented.

In an embodiment of the present invention, the topical composition independently includes about 0.01% to 5% by weight of each active pharmaceutical ingredient (API). For example, the composition can independently include, 0.01%, 0.025%, 0.05%, 0.1%, 0.2%, 0.25%, 0.3%, 0.4%, 0.5%, 0.6%, 0.7%, 0.8%, 0.9%, 1%, 1.5%, 2%, 2.5%, 3%, 4%, 5%, or 10% by weight, of each active pharmaceutical ingredient (API).

The topical compositions of the invention are topically applied directly to the affected area in any conventional manner well known in the art, e.g., by dropper or applicator stick, as a mist via an aerosol applicator, via an intradermal or transdermal patch, or by simply spreading a formulation of the invention onto the affected area with fingers. Generally the amount of a topical formulation of the invention applied to the affected skin area ranges from about 0.1 mg/cm² of skin surface area to about 5 g/cm², preferably, 0.2 mg/cm² to about 1.0 g/cm² of skin surface area. Typically, one to four applications per day are recommended during the term of treatment.

The topical formulations of the invention can be filled and packaged into a plastic squeeze bottle or tube. Suitable container-closure systems for packaging a topical formulation of the invention are commercially available for example, from Wheaton Plastic Products, 1 101 Wheaton Avenue, Millville, N.J. 08332.

Preferably, instructions are packaged with the formulations of the invention, for example, a pamphlet or package label. The labeling instructions explain how to administer topical formulations of the invention, in an amount and for a period of time sufficient to prevent or inhibit skin thickening and signs and/or symptoms associated therewith. Preferably, the label includes the pharmacology, drug resistance, pharmacokinetics, absorption, bioavailability, and contraindications.

This invention will be better understood by reference to the non-limiting examples that follow, but those skilled in the art will readily appreciate that the examples are only illustrative of the invention as described more fully in the claims which follow thereafter.

### PROPHETIC EXAMPLES

Prophetic examples 1-10 below can be readily carried out, employing techniques, materials, and equipment readily known to those reasonably skilled in the art. Examples 1-14 provide for relatively long acting topical formulations that include an NSAID, in combination with one or more additional active pharmaceutical ingredients (APIs). Further, examples 1-9 and 11-14 provide for suitable prescription (Rx) drug products, and example 10 provides for a suitable over-the-counter (OTC) drug product.

### Example 1

NSAID (microencapsulated diclofenac, 0.1-5 wt. %) and antibacterial (benzoyl peroxide, 2.5-10 wt. %).

### Example 2

NSAID (microencapsulated indomethacin, 0.1-5 wt. %) and antibacterial (benzoyl peroxide, 2.5-10 wt. %).

### Example 3

NSAID (microencapsulated diclofenac, 0.1-5% wt. %) and retinoid (mircoencapsulated Retin-A™ (trentinoin), 0.04-0.1 wt. %).

### Example 4

NSAID (microencapsulated diclofenac, 5 wt. %) and retinoid (adapalene, 0.1-0.3 wt. %).

### Example 5

NSAID (microencapsulated diclofenac, 0.1-5 wt. %), antibacterial (benzoyl peroxide, 2.5-10 wt. %), and antibiotic (clindamycin, 1-2 wt. %).

### Example 6

NSAID (vehicle gel diclofenac, 5 wt. %), antibacterial (benzoyl peroxide, 2.5-10 wt. %), and antibiotic (erythromycin, 2 wt. %).

### Example 7

NSAID (vehicle gel diclofenac, 5 wt. %) and retinoid (adapalene, 0.1-0.3 wt. %).

### Example 8

NSAID (vehicle gel diclofenac, 5 wt. %) and antibacterial (benzoyl peroxide, 2.5-10 wt. %).

### Example 9

NSAID (vehicle gel diclofenac, 5 wt. %), antibacterial (benzoyl peroxide, 2.5-10 wt. %), and retinoid (adapalene, 0.1-0.3 wt. %).

### Example 10

NSAID (microencapsulated Ibuprofen, 0.1-15 wt. %), antibacterial (benzoyl peroxide, 2.5 wt. %) and retinoid (retinaldehyde, [0.1-0.5 wt. %).

### Example 11

NSAID (vehicle gel diclofenac, 5 wt. %), antibiotic (clindamycin 1-2 wt. %), and retinoid (adapalene, 0.1-0.3 wt. %).

### Example 12

NSAID (microencapsulated diclofenac 5 wt. %) topically applied within 24 hours of phototherapy UVA/UVB (session approximately 30-90 minutes).

### Example 13

NSAID (microencapsulated diclofenac 5 wt. %) topically applied within 24 hours of oral spironolactone (50 mg daily).

### Example 14

NSAID (microencapsulated diclofenac 5 wt. %) topically applied within 24 hours of oral retinoid (isotretinoin 0.025-0.1 wt. %).

### EXAMPLES

Examples 15-20 below were carried out, employing techniques, materials, and equipment readily known to those reasonably skilled in the art. Examples 15-16 provide for relatively long acting topical formulations, that are suitable prescription (Rx) or over-the-counter (OTC) drug products, which include one or more active pharmaceutical ingredients (APIs). Further, the human clinical studies in Examples 17-20 provide for the utility (e.g., safety and effectiveness) of the topical formulations described herein.

The clindamycin gel used was Clindac-A® (clindamycin phosphate, 1%) topical gel USP, manufactured by Galderma.

The benzoyl peroxide gel used was Benzac AC® (benzoyl peroxide, 2.5%) topical gel, manufactured by Galderma.

The retinoid cream used was Retino-A® (tretinoin, 0.025%) topical cream USP, manufactured by Johnson and Johnson.

The liposomal diclofenac sodium, 1% gel used is the formulation prepared in Example 15.

Liposomes were prepared by rotary evaporation. Soy phospholipid and cholesterol were dissolved in chloroform (5% w/w) in a glass flask. The solvent was removed by rotary evaporation at 90 rpm for 15 minutes at 45° C. and placed under vacuum. After drying, the lipid film was hydrated with 1% diclofenac in a 20% ethanolic solution. Resulting vesicles were then sonicated by the probe method and extruded through 200 nm filters.

### Example 15

A 1 wt. % diclofenac sodium liposomal gel was prepared from the following substances:

| Ingredient | Amount (mg) | Amount (wt. %) |
|---|---|---|
| Diclofenac Sodium | 350 mg | 1% |
| Phospholipid | 675 mg | 1.93% |
| Cholesterol | 75 mg | 0.21% |
| Ethanol | 6,685 mg | 19.1% |
| Carbopol gel | 525 mg | 1.50% |
| Distilled water | 26,690 mg | 76.26% |
| TOTAL | 35,000 mg | 100% |

### Example 16

A 1 wt. % diclofenac sodium and 1 wt. % clindamycin phosphate liposomal gel was prepared from the following substances:

| Ingredient | Amount (mg) | Amount (wt. %) |
|---|---|---|
| Clindamycin phosphate | 350 mg | 1% |
| Diclofenac Sodium | 350 mg | 1% |
| Phospholipid | 675 mg | 1.93% |
| Cholesterol | 75 mg | 0.21% |
| Ethanol | 6,685 mg | 19.1% |
| Carbopol gel | 525 mg | 1.50% |
| Distilled water | 26,340 mg | 75.26% |
| TOTAL | 35,000 mg | 100% |

### Example 17

### Human Clinical Studies

A healthy human volunteer, 19 year old patient, afflicted with moderate acne vulgaris presented with eight inflammatory acne lesions on the right side of her face and two inflammatory lesions on the left side of her face, along with five comedonal/non-inflammatory lesions on either side of her face. Informed consent was obtained. She was initiated on clindamycin phosphate, 1% topical gel to both sides of her face. Liposomal diclofenac sodium, 1% gel was mixed with the clindamycin phosphate, 1% topical gel and applied to the right side of her face once daily. Within 24 hours, there was an approximately 50% reduction in the average size of the inflammatory lesions on the right side of her face. Within 72 hours, all inflammatory lesions on the right side of her face had decreased further in size and four had a flattened morphology. However, at 72 hours the two inflammatory lesions on the left side (control) of her face had not changed significantly in morphology. With continued once daily application, she developed only one additional inflammatory lesion in the following 3 weeks on the intervention side.

### Example 18

### Human Clinical Studies

A healthy 21 year old volunteer male with severe acne vulgaris presented for evaluation and treatment. Informed consent was obtained and the patient was initiated on benzoyl peroxide, 2.5% topical gel, twice daily, to the left side of his face. On the right side of his face he applied liposomal diclofenac sodium, 1% gel, twice daily. On the left side, he had five inflammatory lesions, and seven inflammatory lesions on the right side. He had ten comedones on the right side and twelve on the left side. Within 24 hours of application, there was a reduction in the peri-lesional erythema for all inflammatory lesions on the right side. There was a reduction in size and, over the next 72-96 hours, a flattening of the inflammatory lesions and mild peeling noted on the right side. On the left side, however, the number of lesions increased to eight, whereas each lesion showing a more papular morphology. No new lesions appeared on the right side in the following 3 weeks, while the left side had five additional new inflammatory lesions in that same time period.

### Example 19

### Human Clinical Studies

A healthy 18 year old patient presented with predominantly comedonal acne. He was initiated on tretinoin, 0.025% topical cream to both sides of the face at night, and liposomal diclofenac sodium, 1% gel to the left side of the face once daily. Approximately seven days after starting treatment, there was a decrease in the number of comedonal lesions on the intervention side from ten to eight; this number further decreased over the next 14 days to four. On the right side however, the number of comedones remained steady at twelve, decreasing only after four weeks. Surprisingly, this suggests that non-inflammatory lesions may also respond to liposomal diclofenac sodium, 1% gel, when combined with standard acne treatment.

### Example 20

### Human Clinical Studies

A 21 year old patient presented with severe acne, consisting of inflammatory lesions (eight on the right and nine on the left) and non-inflammatory lesions (fourteen right and twelve on the left). She was initiated on clindamycin phosphate, 1% topical gel to both sides of the face, twice daily and encapsulated, liposomal diclofenac sodium, 1% gel to the right side of the face, twice daily. An immediate reduction was noted in the size of inflammatory lesions within 72 hours on the right/intervention side, and by the end of the week all eight inflammatory lesions had flattened. In contrast, the lesions on the left side continued to progress and grow into papules and nodules, accompanied by erythema. By week two, the number of comedones on the right had decreased to six whereas the left side had ten, with no new lesions, whereas the number of original comedones on the left side had decreased to ten, with several new lesions formed.

### Summary of Examples 15-20:

Several promising and unexpected results were identified as a result of these treatment regimens. Compared to the control, the intervention side showed a significant decrease in new lesion formation. Diclofenac sodium has been found to previously exhibit anti-bacterial activity against gram-negative organisms^{1,2} including species E. Coli and L. monocytogenes due to its ability to inhibit DNA synthesis, as well as its ability to act as a “helper” compound by anti-plasmid activity when administered in conjunction with streptomycin. These clinical findings suggest that diclofenac may possess activity against Propionibacterium acnes, the bacterial species commonly implicated in acne lesions, especially when a controlled release form is combined with other antibiotics such as clindamycin or antibacterials such as benzoyl peroxide. ¹Eur J Clin Microbiol Infect Dis. 2009 August; 28(8):881-91. doi: 10.1007/s10096-009-0739-z. Epub 2009 Apr. 28. The anti-inflammatory non-antibiotic helper compound diclofenac: an antibacterial drug target. Mazumdar K1, Dastidar S G, Park J H, Dutta N K.²Annals of Clinical Microbiology and Antimicrobials 2011, 10:30 doi:10.1186/1476-0711-10-30. Alterations in the transcriptome and antibiotic susceptibility of Staphylococcus aureus grown in the presence of diclofenac. James T Riordan et al.

Also, due to the strong anti-inflammatory properties of diclofenac, other compounds such as retinoids, antibiotics and antibacterials can penetrate deeper in the tissue and remain within the lower layers of the dermis. This allows these compounds to have prolonged activity at the sites where they will be most effective in acne treatment, i.e., the base of the follicles within the dermis. It was noted that on the side where microencapsulated diclofenac was administered, any new lesions that did form did not progress to nodules or cysts, but resolved from papules to macules (flat lesions) or closed comedones.

Another surprising effect noted was that no initial acne flare-up was seen in patients on retinoids. A similar result has been previously described with the use of corticosteroids with retinoid medications; however, corticosteroids are generally contraindicated in acne since they can themselves cause acne, can cause skin atrophy and striae, and can cause rebound acne when stopped. Sustained release NSAID combinations with retinoids may provide a way to ameliorate any initial flare-ups seen with topical retinoids without the harmful side effects of corticosteroid therapy.

Another benefit of reduced skin irritation and rapid response that characterizes the delivery of a controlled release strong anti-inflammatory in acne is the reduction in scarring, post-inflammatory hypopigmentation and hyperpigmentation that occurs in the aftermath of an acne breakout. One patient demonstrated a propensity to scarring and five lesions were treated with liposomal diclofenac and clindamycin. Within one week all lesions had flattened, and at the time of six week follow up, no scar formation or early fibrosis had been noted. Subjective evaluations provided by the patients indicated the majority perceived a decrease in secondary dyspigmentation.

Additionally, subjective evaluation by patients regarding the rapid reduction in inflammatory lesions and reduced new lesion formation revealed multiple psychological benefits. With quicker results, patients felt more compelled to be compliant with NSAID therapy. Patients had an improved self-image.

Yet another unexpected result of using controlled release forms of NSAID medications was a reduction in closed and open comedones, as compared to control. Although these lesions are considered to be non-inflammatory, there may be an inflammatory component to the keratinocyte proliferation that is responsible for the lesions. This may respond to the control of inflammation provided by the NSAID. Alternatively, as described above, decreased inflammation from controlled release NSAIDs may allow for increased penetration of standard anti-acne regimens, improving their efficacy.

Table 1 is a summary of the total lesions count in 10 patients in intervention vs control side. The ten patients were a mixed group of clindamycin treatment, benzoyl peroxide treatment, and retinoid treatment.

TABLE 1
Lesion progress for ten patients treated for a six week period-
Split face evaluation

| | SUMMARY | | | | | | | | | |
|---|---|---|---|---|---|---|---|---|---|---|
| | 72 HOUR | | Day 7 | | Day 14 | | Day 28 | | Week 6 | |
| | Clinical observation | | | | | | | | | |
| | Intervention(I) Control - (C) | | | | | | | | | |
| | I | C | I | C | I | C | I | C | I | C |
| Average % reduction in | 30.6 | 7.2 | 49.8 | −16.7 | 76 | 9.1 | 80.39 | 28.3 | 82.15 | 30.9 |
| inflammatory lesions from | | | | | | | | | | |
| baseline | | | | | | | | | | |
| Average % reduction in | 0 | 3.8 | 1.1 | 2.2 | 27.7 | 0.2 | 57 | 16.3 | 62.2 | 20.2 |
| non-inflammatory lesions | | | | | | | | | | |
| from baseline | | | | | | | | | | |
| New inflammatory lesions | 2 | 6 | 2 | 8 | 3 | 14 | 2 | 9 | 3 | 10 |
| formed | | | | | | | | | | |
| New non-inflammatory | 3 | 2 | 2 | 7 | 3 | 10 | 4 | 14 | 4 | 11 |
| lesions formed | | | | | | | | | | |

### ENUMERATED EMBODIMENTS

Specific enumerated embodiments [1] to [149] provided below are for illustration purposes only, and do not otherwise limit the scope of the invention, as defined by the claims. These enumerated embodiments encompass all combinations, sub-combinations, and multiply referenced (e.g., multiply dependent) combinations described therein.

[1.] The present invention provides for a topical dermatological composition including a pharmaceutically acceptable medium and at least one nonsteroidal anti-inflammatory drug (NSAID) in the form of a microencapsulation, liposome, niosome, microsponge, microemulsion, micronized particles, microsphere, SLN, hydrogel, aerosol, or fullerene.
[2.] The present invention also provides for a topical dermatological composition including:

(i) a pharmaceutically acceptable medium;

(ii) a nonsteroidal anti-inflammatory drug (NSAID);

(iii) optionally an antibiotic;

(iv) optionally an antibacterial agent;

(v) optionally a retinoid;

(vi) optionally an anti-androgenic agent;

(vii) optionally a folate;

(viii) optionally a keratolytic agent;

(ix) optionally at least one of azelaic acid, alpha hydroxy acid, trichloroacetic acid, lactic acid, ethanol, resorcinol, sulfur, sodium sulfacetamide, ivermectin, and glycolic acid; and

(x) optionally at least one penetrating agent;

wherein at least one of (a) and (b) is satisfied:

(a) any one or more of the components (ii)-(x) is optionally microencapsulated, and

(b) at least one (e.g., 1, 2, 3, 4, 5, 6, 7, 8, 9, etc.) of (iii)-(ix) is present.

[3.] The present invention also provides for a topical dermatological composition of any one of the preceding embodiments, wherein the microencapsulation is a single, double, or triple layer encapsulation.
[4.] The present invention also provides for a topical dermatological composition of any one of the preceding embodiments, wherein the microencapsulation is carried out employing microparticles having a mean average particle size diameter of less than about 300 micrometers.
[5.] The present invention also provides for a topical dermatological composition of any one of the preceding embodiments, wherein the nonsteroidal anti-inflammatory drug (NSAID) includes at least one of an:

(i) N-arylanthranilic acid;

(ii) 2-arylpropionic acid;

(iii) arylalkanoic acid; and

(iv) cyclooxygenase (COX) inhibitor.

[6.] The present invention also provides for a topical dermatological composition of any one of the preceding embodiments, wherein the nonsteroidal anti-inflammatory drug (NSAID) includes at least one of flufenamic acid, meclofenamic acid, mefenamic acid, tolfenamic acid, alminoprofen, carprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, naproxen, oxaprozin, pirprofen, suprofen, tiaprofenic acid, aceclofenac, acemetacin, alclofenac, bromfenac, diclofenac, etodolac, indomethacin, nabumetone, oxametacin, proglumetacin, sulindac, and tolmetin.
[7.] The present invention also provides for a topical dermatological composition of any one of the preceding embodiments, wherein the antibiotic includes at least one of erythromycin, metronidazole, clindamycin, doxycycline, minocycline, tetracycline, oxytetracycline, chlortetracycline, dapsone, demeclocycline, and co-trimoxazole.
[8.] The present invention also provides for a topical dermatological composition of any one of the preceding embodiments, wherein the retinoid includes at least one of retinal, retinol, retinal, retinaldehyde tretinoin (retinoic acid, Retin-A), isotretinoin, alitretinoin, etretinate, acitretin, tazarotene, bexarotene and adapalene.
[9.] The present invention also provides for a topical dermatological composition of any one of the preceding embodiments, wherein the anti-androgenic includes at least one of ASC-J9, topical nitric oxide, and an antihistamine.
[10.] The present invention also provides for a topical dermatological composition of any one of the preceding embodiments, wherein the keratolytic agent includes salicylic acid.
[11.] The present invention also provides for a topical dermatological composition of any one of the preceding embodiments, wherein the antibacterial agent includes benzoyl peroxide.
[12.] The present invention also provides for a topical dermatological composition of any one of the preceding embodiments, which is in the form of a cleanser or wash, pledget, solution, suspension, foam, cream, spray, aqueous gel, non-aqueous gel, ointment, emulsion, emulsion-gel, cream-gel, emulsion-gel/cream-gel oil, toner, lotion, aerosol, or topical adhesive patch.
[13.] The present invention also provides for a topical dermatological composition of any one of the preceding embodiments, formulated as a medicament.
[14.] The present invention also provides for a topical dermatological composition of any one of the preceding embodiments, further including one or more additives selected from the group consisting of fragrances, preserving agents, stabilizers, pH regulators, UV-A screening agents, UV-B screening agents, and antioxidants.
[15.] The present invention also provides for a topical dermatological composition of any one of the preceding embodiments, wherein the nonsteroidal anti-inflammatory drug (NSAID) is present in up to about 30 wt. % of the topical dermatological composition.
[16.] The present invention also provides for a topical dermatological composition of any one of the preceding embodiments, wherein the antibiotic is present in up to about 5 wt. % of the topical dermatological composition.
[17.] The present invention also provides for a topical dermatological composition of any one of the preceding embodiments, wherein the antibacterial agent is present in up to about 15 wt. % of the topical dermatological composition.
[18.] The present invention also provides for a topical dermatological composition of any one of the preceding embodiments, wherein the retinoid is present in up to about 0.3 wt. % of the topical dermatological composition.
[19.] The present invention also provides for a topical dermatological composition of any one of the preceding embodiments, wherein the anti-androgenic agent is present in up to about 10 wt. % of the topical dermatological composition.
[20.] The present invention also provides for a topical dermatological composition of any one of the preceding embodiments, wherein the folate is present in up to about 5 wt. % of the topical dermatological composition.
[21.] The present invention also provides for a topical dermatological composition of any one of the preceding embodiments, wherein the keratolytic agent is present in up to about 2 wt. % of the topical dermatological composition.
[22.] The present invention also provides for a topical dermatological composition of any one of the preceding embodiments, wherein the azelaic acid is present in up to about 20 wt. % of the topical dermatological composition.
[23.] The present invention also provides for a topical dermatological composition of any one of the preceding embodiments, wherein the alpha hydroxy acid is present in up to about 10 wt. % of the topical dermatological composition.
[24.] The present invention also provides for a topical dermatological composition of any one of the preceding embodiments, wherein the trichloroacetic acid is present in up to about 80 wt. % of the topical dermatological composition.
[25.] The present invention also provides for a topical dermatological composition of any one of the preceding embodiments, wherein the lactic acid is present in up to about 10 wt. % of the topical dermatological composition.
[26.] The present invention also provides for a topical dermatological composition of any one of the preceding embodiments, wherein the ethanol, or a C₁-C₈ alcohol, is present in up to about 20 wt. % of the topical dermatological composition.
[27.] The present invention also provides for a topical dermatological composition of any one of the preceding embodiments, wherein the resorcinol is present in up to about 0.05 wt. % of the topical dermatological composition.
[28.] The present invention also provides for a topical dermatological composition of any one of the preceding embodiments, wherein the sulfur is present in up to about 20 wt. % of the topical dermatological composition.
[29.] The present invention also provides for a topical dermatological composition of any one of the preceding embodiments, wherein the sodium sulfacetamide is present in up to about 10 wt. % of the topical dermatological composition.
[30.] The present invention also provides for a topical dermatological composition of any one of the preceding embodiments, wherein the glycolic acid is present in up to about 30 wt. % of the topical dermatological composition.
[31.] The present invention also provides for a topical dermatological composition of any one of the preceding embodiments, wherein the (NSAID) in the form of a microencapsulation.
[32.] The present invention also provides for a topical dermatological composition of any one of the preceding embodiments, wherein the (NSAID) in the form of a liposome.
[33.] The present invention also provides for a topical dermatological composition of any one of the preceding embodiments, wherein the wherein the (NSAID) in the form of a noisome.
[34.] The present invention also provides for a topical dermatological composition of any one of the preceding embodiments, wherein the wherein the (NSAID) in the form of a microsponge.
[35.] The present invention also provides for a topical dermatological composition of any one of the preceding embodiments, wherein the wherein the (NSAID) in the form of a microemulsion or microsphere.
[36.] The present invention also provides for a topical dermatological composition of any one of the preceding embodiments, wherein the wherein the (NSAID) in the form of an SLN.
[37.] The present invention also provides for a topical dermatological composition of any one of the preceding embodiments, wherein the wherein the (NSAID) in the form of a hydrogel.
[38.] The present invention also provides for a topical dermatological composition of any one of the preceding embodiments, wherein the wherein the (NSAID) in the form of an aerosol.
[39.] The present invention also provides for a topical dermatological composition of any one of the preceding embodiments, wherein the wherein the (NSAID) is in the form of a fullerene.
[40.] The present invention also provides for a topical dermatological composition of any one of the preceding embodiments, wherein at least one of (iii)-(xi) is present.
[41.] The present invention also provides for a topical dermatological composition of any one of the preceding embodiments, where the antibiotic is present.
[42.] The present invention also provides for a topical dermatological composition of any one of the preceding embodiments, where the antibiotic is microencapsulates.
[43.] The present invention also provides for a present invention also provides for a topical dermatological composition of any one of the preceding embodiments, where the antibiotic is absent.
[44.] The present invention also provides for a topical dermatological composition of any one of the preceding embodiments, where the antibacterial agent is present.
[45.] The present invention also provides for a topical dermatological composition of any one of the preceding embodiments, where the antibacterial agent is present and is microencapsulated.
[46.] The present invention also provides for a topical dermatological composition of any one of the preceding embodiments, where the antibacterial agent is absent.
[47.] The present invention also provides for a topical dermatological composition of any one of the preceding embodiments, where the retinoid is present.
[48.] The present invention also provides for a topical dermatological composition of any one of the preceding embodiments, where the retinoid is present and is microencapsulated.
[49.] The present invention also provides for a topical dermatological composition of any one of the preceding embodiments, where the retinoid is absent.
[50.] The present invention also provides for a topical dermatological composition of any one of the preceding embodiments, where the anti-androgenic agent is present.
[51.] The present invention also provides for a topical dermatological composition of any one of the preceding embodiments, where the anti-androgenic agent is present and is microencapsulated.
[52.] The present invention also provides for a topical dermatological composition of any one of the preceding embodiments, where the anti-androgenic agent is absent.
[53.] The present invention also provides for a topical dermatological composition of any one of the preceding embodiments, where the folate is present.
[54.] The present invention also provides for a topical dermatological composition of any one of the preceding embodiments, where the folate is present and is microencapsulated.
[55.] The present invention also provides for a topical dermatological composition of any one of the preceding embodiments, where the folate is absent.
[56.] The present invention also provides for a topical dermatological composition of any one of the preceding embodiments, where the keratolytic agent is present.
[57.] The present invention also provides for a topical dermatological composition of any one of the preceding embodiments, where the keratolytic agent is present and is microencapsulated.
[58.] The present invention also provides for a topical dermatological composition of any one of the preceding embodiments, where the keratolytic agent is absent.
[59.] The present invention also provides for a topical dermatological composition of any one of the preceding embodiments, where the azelaic acid is present.
[60.] The present invention also provides for a topical dermatological composition of any one of the preceding embodiments, where the azelaic acid is present and is microencapsulated.
[61.] The present invention also provides for a topical dermatological composition of any one of the preceding embodiments, where the azelaic acid is absent.
[62.] The present invention also provides for a topical dermatological composition of any one of the preceding embodiments, where the alpha hydroxy acid is present.
[63.] The present invention also provides for a topical dermatological composition of any one of the preceding embodiments, where the alpha hydroxy acid is present and is microencapsulated.
[64.] The present invention also provides for a topical dermatological composition of any one of the preceding embodiments, where the alpha hydroxy acid is absent.
[65.] The present invention also provides for a topical dermatological composition of any one of the preceding embodiments, where the trichloroacetic acid is present.
[66.] The present invention also provides for a topical dermatological composition of any one of the preceding embodiments, where the trichloroacetic acid is present and is microencapsulated.
[67.] The present invention also provides for a topical dermatological composition of any one of the preceding embodiments, where the trichloroacetic acid is absent.
[68.] The present invention also provides for a topical dermatological composition of any one of the preceding embodiments, where the lactic acid is present.
[69.] The present invention also provides for a topical dermatological composition of any one of the preceding embodiments, where the lactic acid is present and is microencapsulated.
[70.] The present invention also provides for a topical dermatological composition of any one of the preceding embodiments, where the lactic acid is absent.
[71.] The present invention also provides for a topical dermatological composition of any one of the preceding embodiments, where the ethanol is present.
[72.] The present invention also provides for a topical dermatological composition of any one of the preceding embodiments, where the ethanol is present and is microencapsulated.
[73.] The present invention also provides for a topical dermatological composition of any one of the preceding embodiments, where the ethanol is absent.
[74.] The present invention also provides for a topical dermatological composition of any one of the preceding embodiments, where the resorcinol is present.
[75.] The present invention also provides for a topical dermatological composition of any one of the preceding embodiments, where the resorcinol is present and is microencapsulated.
[76.] The present invention also provides for a topical dermatological composition of any one of the preceding embodiments, where the resorcinol is absent.
[77.] The present invention also provides for a topical dermatological composition of any one of the preceding embodiments, where the sulfur is present.
[78.] The present invention also provides for a topical dermatological composition of any one of the preceding embodiments, where the sulfur is present and is microencapsulated.
[79.] The present invention also provides for a topical dermatological composition of any one of the preceding embodiments, where the sulfur is absent.
[80.] The present invention also provides for a topical dermatological composition of any one of the preceding embodiments, where the sodium sulfacetamide is present.
[81.] The present invention also provides for a topical dermatological composition of any one of the preceding embodiments, where the sodium sulfacetamide is present and is microencapsulated.
[82.] The present invention also provides for a topical dermatological composition of any one of the preceding embodiments, where the sodium sulfacetamide is absent.
[83.] The present invention also provides for a topical dermatological composition of any one of the preceding embodiments, where the ivermectin is present.
[84.] The present invention also provides for a topical dermatological composition of any one of the preceding embodiments, where the ivermectin is present and is microencapsulated.
[85.] The present invention also provides for a topical dermatological composition of any one of the preceding embodiments, where the ivermectin is absent.
[86.] The present invention also provides for a topical dermatological composition of any one of the preceding embodiments, where the glycolic acid is present.
[87.] The present invention also provides for a topical dermatological composition of any one of the preceding embodiments, where the glycolic acid is present and is microencapsulated.
[88.] The present invention also provides for a topical dermatological composition of any one of the preceding embodiments, where the glycolic acid is absent.
[89.] The present invention also provides for a topical dermatological composition of any one of the preceding embodiments, where the penetrating agent is present.
[90.] The present invention also provides for a topical dermatological composition of any one of the preceding embodiments, where the penetrating agent is present and is microencapsulated.
[91.] The present invention also provides for a topical dermatological composition of any one of the preceding embodiments, where the penetrating agent is absent.
[92.] The present invention also provides for a method of treating a topical skin disorder in a human patient, the method including topically administering to a human patient in need thereof, the topical dermatological composition of any one of the preceding embodiments, in an amount and for a period of time effective to treat the topical skin disorder.
[93.] The present invention also provides for a method of treating acne vulgaris in a human patient, the method including topically administering to a human patient in need thereof, the topical dermatological composition of any one of the preceding embodiments, in an amount and for a period of time effective to treat the acne vulgaris.
[94.] The present invention also provides for a method of any one of the preceding embodiments, wherein the administration occurs in combination with a chemical peel to reduce associated inflammatory response.
[95.] The present invention also provides for a method of any one of the preceding embodiments, wherein the administration occurs in combination with the oral administration of at least one of an oral anti-androgen (e.g., spironolactone, cimetidine) and an oral contraceptive.
[96.] The present invention also provides for a method of any one of the preceding embodiments, wherein the administration occurs in combination with at least one of photodynamic therapy, blue or red light phototherapy, and phototherapy with UVA or UVB therapy, to enhance efficacy.
[97.] The present invention also provides for a method of any one of the preceding embodiments, wherein the administration occurs in combination with the oral administration of an oral antibiotic (e.g., doxycycline or minocycline).
[98.] The present invention also provides for a method of any one of the preceding embodiments, wherein the administration occurs in combination with intense pulsed light.
[99.] The present invention also provides for a method of any one of the preceding embodiments, wherein the human patient is an adolescent, between the ages of about 10 and 18.
[100.] The present invention also provides for a method of any one of the preceding embodiments, wherein the treating of the acne vulgaris includes treating at least one of seborrhea, comedones, papules, pustules, nodules, and pimples.
[101.] The present invention also provides for a method of any one of the preceding embodiments, further including prior to administrating the topical dermatological composition, washing and drying the affected area.
[102.] The present invention also provides for a method of any one of the preceding embodiments, further including prior to administrating the topical dermatological composition, washing with a topical cleanser and subsequently drying the affected area.
[103.] The present invention also provides for a method of any one of the preceding embodiments, further including prior to administrating the topical dermatological composition, washing with at least one of soap and water, toner, lotion, pleglet, and foam, and subsequently drying the affected area.
[104.] The present invention also provides for a method of any one of the preceding embodiments, wherein the acne vulgaris is mild to severe acne vulgaris.
[105.] The present invention also provides for a method of any one of the preceding embodiments, wherein the topical skin disorder includes at least one of acne vulgaris, comedonic or polymorphic acne, nodulocystic acne, acne conglobata, senile acne or secondary acne, acne lesions, cystic acne, acne atrophica, bromide acne, chlorine acne, acne cosmetica, acne detergicans, epidemic acne, acne estivalis, acne fulminans, halogen acne, acne indurata, iodide acne, acne keloid, acne mechanica, acne papulosa, pomade acne, premenstrual acne, acne pustulosa, hidradenitis suppurativa, keratosis pilaris, acne rosacea, acne scorbutica, acne scrofulosorum, acne urticata, acne varioliformis, acne venenata, propionic acne, acne excoriee, pseudofolliculitis barbae, gram negative acne, steroid acne, pregnancy related acne, folliculitis, odulocystic acne, rosacea, common acne, seborrheic dermatitis, keratosis follicularis, perioral dermatitis, transient acantholytic dermatitis, dermatosis, focal acantholytic dyskeratosis, acne necrotica milliaris, seborrhea, comedones, papules, pustules, nodules, and pimples.
[106.] The present invention also provides for a method of any one of the preceding embodiments, wherein the topical administration occurs at least once per week.
[107.] The present invention also provides for a method of any one of the preceding embodiments, wherein the topical administration occurs for a period of time of at least six (6) days.
[108.] The present invention also provides for a method of any one of the preceding embodiments, wherein the topical administration is carried out such that a fixed-dose of each of the nonsteroidal anti-inflammatory drug (NSAID), antibiotic, and antibacterial agent is topically administered.
[109.] The present invention also provides for a method of any one of the preceding embodiments, wherein the topical dermatological composition further includes tea tree oil.
[110.] The present invention also provides for a method of any one of the preceding embodiments, wherein the topical dermatological composition further includes almond extract.
[111.] The present invention also provides for a method of treating acne vulgaris in a human patient, the method includes topically administering to a human patient in need thereof, the topical dermatological composition of any one of the preceding embodiments, wherein the administration occurs in combination with the oral administration of at least one of an oral antibiotic (e.g. erythromycin, metronidazole, co-trimoxazole, clindamycin, doxycycline, minocycline, tetracycline, oxytetracycline, chlortetracycline, dapsone, and demeclocycline).
[112.] The present invention also provides for a method of any one of the preceding embodiments, wherein the administration is carried out such that the nonsteroidal anti-inflammatory drug (NSAID) and the at least one of components (i)-(vii) are co-administered, together, at the same time.
[113.] The present invention also provides for a method of any one of the preceding embodiments, wherein the administration is carried out such that the nonsteroidal anti-inflammatory drug (NSAID) and the at least one of components (i)-(vii) are concurrently administered, ad seriatim, in a serial fashion.
[114.] The present invention also provides for a method of any one of the preceding embodiments, wherein the nonsteroidal anti-inflammatory drug (NSAID), and optionally one or more of components (i)-(vii), are microencapsulated.
[115.] The present invention also provides for a method of any one of the preceding embodiments, wherein the nonsteroidal anti-inflammatory drug (NSAID), and optionally one or more of components (i)-(vii), are at least partially (e.g., at least about 10 wt. %, 20 wt. %, 30 wt. %, 40 wt. %, 50 wt. %, 60 wt. %, 70 wt. %, 80 wt. %, or 90 wt. %) microencapsulated.
[116.] The present invention also provides for a method of any one of the preceding embodiments, wherein the nonsteroidal anti-inflammatory drug (NSAID), and optionally one or more of components (i)-(vii), are completely (e.g., about 100 wt. %) microencapsulated.
[117.] The present invention also provides for a method of any one of the preceding embodiments, wherein the nonsteroidal anti-inflammatory drug (NSAID) is at least partially (e.g., at least about 10 wt. %, 20 wt. %, 30 wt. %, 40 wt. %, 50 wt. %, 60 wt. %, 70 wt. %, 80 wt. %, or 90 wt. %) microencapsulated.
[118.] The present invention also provides for a method of any one of the preceding embodiments, wherein the nonsteroidal anti-inflammatory drug (NSAID) is completely (e.g., about 100 wt. %) microencapsulated.
[119.] The present invention also provides for a method of any one of the preceding embodiments, wherein the topical composition is combined with an oral retinoid (e.g. isotretinoin).
[120.] The present invention also provides for a topical dermatological composition that includes diclofenac and clindamycin, in a pharmaceutically acceptable medium, wherein at least one of the diclofenac and clindamycin is microencapsulated.
[121.] The present invention also provides for a topical dermatological composition of the above embodiment, wherein the diclofenac is in the form of liposome, niosome, microsponge, microemulsion, micronized particle, microsphere, SLN, hydrogel, aerosol, or fullerene.
[122.] The present invention also provides for a topical dermatological composition of any one of the above embodiments, wherein the clindamycin is in the form of liposome, niosome, microsponge, microemulsion, micronized particles, microsphere, SLN, hydrogel, aerosol, or fullerene.
[123.] The present invention also provides for a topical dermatological composition of any one of the above embodiments, wherein both of the diclofenac and clindamycin are microencapsulated.
[124.] The present invention also provides for a topical dermatological composition of any one of the above embodiments, wherein both of the diclofenac and clindamycin are independently in the form of liposome, niosome, microsponge, microemulsion, micronized particles, microsphere, SLN, hydrogel, aerosol, or fullerene.
[125.] The present invention also provides for a topical dermatological composition of any one of the above embodiments, further including at least one of (i)-(viii):

(i) antibiotic;

(ii) antibacterial agent;

(iii) retinoid;

(iv) anti-androgenic agent;

(v) folate;

(vi) keratolytic agent;

(vii) at least one of azelaic acid, alpha hydroxy acid, trichloroacetic acid, lactic acid, ethanol, resorcinol, sulfur, sodium sulfacetamide, ivermectin, and glycolic acid; and

(viii) penetrating agent.

[126.] The present invention also provides for a topical dermatological composition of any one of the above embodiments, wherein the retinoid is present and includes at least one of retinal, retinol, retinal, retinaldehyde tretinoin (retinoic acid, Retin-A), isotretinoin, alitretinoin, etretinate, acitretin, tazarotene, bexarotene and adapalene.
[127.] The present invention also provides for a topical dermatological composition of any one of the above embodiments, wherein the antibacterial agent is present and includes benzoyl peroxide.
[128.] The present invention also provides for a topical dermatological composition of any one of the above embodiments, which is in the form of a cleanser or wash, pledglet, solution, suspension, foam, cream, spray, aqueous gel, non-aqueous gel, ointment, emulsion, emulsion-gel, cream-gel, emulsion-gel/cream-gel oil, toner, lotion, aerosol, or topical adhesive patch.
[129.] The present invention also provides for a topical dermatological composition of any one of the above embodiments, wherein the diclofenac is present in up to about 10 wt. % of the topical dermatological composition.
[130.] The present invention also provides for a topical dermatological composition of any one of the above embodiments, wherein the clindamycin is present in up to about 5 wt. % of the topical dermatological composition.
[131.] The present invention also provides for a topical dermatological composition of any one of the above embodiments, wherein the antibiotic is present, and is present in an amount up to about 5 wt. % of the topical dermatological composition.
[132.] The present invention also provides for a topical dermatological composition of any one of the above embodiments, wherein the antibacterial agent is present, and is present in an amount up to about 15 wt. % of the topical dermatological composition.
[133.] The present invention also provides for a topical dermatological composition of any one of the above embodiments, wherein the retinoid is present, and is present in an amount up to about 0.3 wt. % of the topical dermatological composition.
[134.] The present invention also provides for a topical dermatological composition of any one of the above embodiments, wherein the diclofenac, and optionally one or more of components (i)-(vii):

(i) antibiotic;

(ii) antibacterial agent;

(iii) retinoid;

(iv) anti-androgenic agent;

(v) folate;

(vi) keratolytic agent; and

(vii) at least one of azelaic acid, alpha hydroxy acid, trichloroacetic acid, lactic acid, ethanol, resorcinol, sulfur, sodium sulfacetamide, ivermectin, and glycolic acid;

is microencapsulated.

[135.] The present invention also provides for a topical dermatological composition of any one of the above embodiments, wherein the diclofenac is at least partially microencapsulated.
[136.] The present invention also provides for a topical dermatological composition of any one of the above embodiments, wherein the clindamycin is at least partially microencapsulated.
[137.] The present invention also provides for a method of treating acne vulgaris in a human patient, the method including topically administering to a human patient in need thereof, a topical dermatological composition that includes diclofenac and clindamycin, in a pharmaceutically acceptable medium, wherein at least one of the diclofenac and clindamycin is microencapsulated, the dermatological composition is administered in an amount and for a period of time effective to treat the acne vulgaris.
[138.] The present invention also provides for a method of embodiment [137], wherein the administration is carried out such that the diclofenac and clindamycin are concurrently administered, together, with the at least one of components (i)-(vii):

(i) antibiotic;

(ii) antibacterial agent;

(iii) retinoid;

(iv) anti-androgenic agent;

(v) folate;

(vi) keratolytic agent; and

(vii) at least one of azelaic acid, alpha hydroxy acid, trichloroacetic acid, lactic acid, ethanol, resorcinol, sulfur, sodium sulfacetamide, ivermectin, and glycolic acid.

[139.] The present invention also provides for a method of embodiment [137], wherein the administration is carried out such that the diclofenac and clindamycin are administered, ad seriatim, with the at least one of components (i)-(vii):

(i) antibiotic;

(ii) antibacterial agent;

(iii) retinoid;

(iv) anti-androgenic agent;

(v) folate;

(vi) keratolytic agent; and

(vii) at least one of azelaic acid, alpha hydroxy acid, trichloroacetic acid, lactic acid, ethanol, resorcinol, sulfur, sodium sulfacetamide, ivermectin, and glycolic acid.

[140.] The present invention also provides for a method of any one of embodiments [137]-[139], wherein the topical administration occurs at least once per week.
[141.] The present invention also provides for a method of any one of embodiments [137]-[140], wherein the acne vulgaris is mild to severe acne vulgaris.
[142.] The present invention also provides for a method of any one of embodiments [137]-[141], wherein the topical dermatological composition is concurrently administered with at least one of an oral antibiotic, oral anti-androgen, and oral retinoid.
[143.] The present invention also provides for a method of any one of embodiments [137]-[142], where the topical dermatological composition includes diclofenac and clindamycin that are both independently microencapsulated.
[144.] The present invention also provides for a method of any one of embodiments [137]-[143], where the topical dermatological composition further includes at least one of:

(i) antibiotic;

(ii) antibacterial agent;

(iii) retinoid;

(iv) anti-androgenic agent;

(v) folate;

(vi) keratolytic agent;

(vii) at least one of azelaic acid, alpha hydroxy acid, trichloroacetic acid, lactic acid, ethanol, resorcinol, sulfur, sodium sulfacetamide, ivermectin, and glycolic acid; and

(viii) penetrating agent.

[145.] The present invention also provides for a method of any one of embodiments [137]-[144], where the topical dermatological composition includes diclofenac that is microencapsulated in a liposome.
[146.] The present invention also provides for a method of any one of embodiments [137]-[145], where the topical dermatological composition includes clindamycin that is microencapsulated in a liposome.
[147.] The present invention also provides for a method of any one of embodiments [137]-[146], where the topical dermatological composition includes diclofenac present in up to about 10 wt. % of the topical dermatological composition.
[148.] The present invention also provides for a method of any one of embodiments [137]-[147], where the topical dermatological composition includes clindamycin present in up to about 5 wt. % of the topical dermatological composition.
[149.] The present invention also provides for a method of treating acne vulgaris in a human patient, the method includes topically administering to a human patient in need thereof, a topical dermatological composition that includes, in a pharmaceutically acceptable medium:

diclofenac, present in up to about 10 wt. % of the topical dermatological composition; and

clindamycin, present in up to about 5 wt. % of the topical dermatological composition;

the topical dermatological composition further including at least one of (i)-(viii):

(i) antibiotic;

(ii) antibacterial agent;

(iii) retinoid;

(iv) anti-androgenic agent;

(v) folate;

(vi) keratolytic agent;

(vii) at least one of azelaic acid, alpha hydroxy acid, trichloroacetic acid, lactic acid, ethanol, resorcinol, sulfur, sodium sulfacetamide, ivermectin, and glycolic acid; and

(viii) penetrating agent;

wherein at least one of the diclofenac and clindamycin is at least partially microencapsulated in the form of liposome;

wherein the dermatological composition is administered in an amount and for a period of time effective to treat the acne vulgaris.

1. A topical dermatological composition comprising diclofenac and clindamycin, in a pharmaceutically acceptable medium, wherein at least one of the diclofenac and clindamycin is microencapsulated.

2. The topical dermatological composition of claim 1, wherein the diclofenac is in the form of liposome, niosome, microsponge, microemulsion, micronized particle, microsphere, SLN, hydrogel, aerosol, or fullerene.

3. The topical dermatological composition of claim 1, wherein the clindamycin is in the form of liposome, niosome, microsponge, microemulsion, micronized particles, microsphere, SLN, hydrogel, aerosol, or fullerene.

4. The topical dermatological composition of claim 1, wherein both of the diclofenac and clindamycin are microencapsulated.

5. The topical dermatological composition of claim 1, wherein both of the diclofenac and clindamycin are independently in the form of liposome, niosome, microsponge, microemulsion, micronized particles, microsphere, SLN, hydrogel, aerosol, or fullerene.

6. The topical dermatological composition of claim 1, further comprising at least one of (i)-(viii):
(i) antibiotic;
(ii) antibacterial agent;
(iii) retinoid;
(iv) anti-androgenic agent;
(v) folate;
(vi) keratolytic agent;
(vii) at least one of azelaic acid, alpha hydroxy acid, trichloroacetic acid, lactic acid, ethanol, resorcinol, sulfur, sodium sulfacetamide, ivermectin, and glycolic acid; and
(viii) penetrating agent.

7. The topical dermatological composition of claim 6, wherein the retinoid is present and comprises at least one of retinal, retinol, retinal, retinaldehyde tretinoin (retinoic acid, Retin-A), isotretinoin, alitretinoin, etretinate, acitretin, tazarotene, bexarotene and adapalene.

8. The topical dermatological composition of claim 6, wherein the antibacterial agent is present and comprises benzoyl peroxide.

9. The topical dermatological composition of claim 6, which is in the form of a cleanser or wash, pledglet, solution, suspension, foam, cream, spray, aqueous gel, non-aqueous gel, ointment, emulsion, emulsion-gel, cream-gel, emulsion-gel/cream-gel oil, toner, lotion, aerosol, or topical adhesive patch.

10. The topical dermatological composition of claim 1, wherein the diclofenac is present in up to about 10 wt. % of the topical dermatological composition.

11. The topical dermatological composition of claim 1, wherein the clindamycin is present in up to about 5 wt. % of the topical dermatological composition.

12. The topical dermatological composition of claim 6, wherein the antibiotic is present, and is present in an amount up to about 5 wt. % of the topical dermatological composition.

13. The topical dermatological composition of claim 6, wherein the antibacterial agent is present, and is present in an amount up to about 15 wt. % of the topical dermatological composition.

14. The topical dermatological composition of claim 6, wherein the retinoid is present, and is present in an amount up to about 0.3 wt. % of the topical dermatological composition.

15. The topical dermatological composition of claim 1, wherein the diclofenac, and optionally one or more of components (i)-(vii):
(i) antibiotic;
(ii) antibacterial agent;
(iii) retinoid;
(iv) anti-androgenic agent;
(v) folate;
(vi) keratolytic agent; and
(vii) at least one of azelaic acid, alpha hydroxy acid, trichloroacetic acid, lactic acid, ethanol, resorcinol, sulfur, sodium sulfacetamide, ivermectin, and glycolic acid;
is microencapsulated.

16. The topical dermatological composition of claim 1, wherein the diclofenac is at least partially microencapsulated.

17. The topical dermatological composition of claim 1, wherein the clindamycin is at least partially microencapsulated.

18. A method of treating acne vulgaris in a human patient, the method comprising topically administering to a human patient in need thereof, a topical dermatological composition comprising diclofenac and clindamycin, in a pharmaceutically acceptable medium, wherein at least one of the diclofenac and clindamycin is microencapsulated, the dermatological composition is administered in an amount and for a period of time effective to treat the acne vulgaris.

19. The method of claim 18, wherein the administration is carried out such that the diclofenac and clindamycin are concurrently administered, together, with the at least one of components (i)-(vii):
(i) antibiotic;
(ii) antibacterial agent;
(iii) retinoid;
(iv) anti-androgenic agent;
(v) folate;
(vi) keratolytic agent; and
(vii) at least one of azelaic acid, alpha hydroxy acid, trichloroacetic acid, lactic acid, ethanol, resorcinol, sulfur, sodium sulfacetamide, ivermectin, and glycolic acid.

20. The method of claim 18, wherein the administration is carried out such that the diclofenac and clindamycin are administered, ad seriatim, with the at least one of components (i)-(vii):
(i) antibiotic;
(ii) antibacterial agent;
(iii) retinoid;
(iv) anti-androgenic agent;
(v) folate;
(vi) keratolytic agent; and
(vii) at least one of azelaic acid, alpha hydroxy acid, trichloroacetic acid, lactic acid, ethanol, resorcinol, sulfur, sodium sulfacetamide, ivermectin, and glycolic acid.

21. The method of claim 18, wherein the topical administration occurs at least once per week.

22. The method of claim 18, wherein the acne vulgaris is mild to severe acne vulgaris.

23. The method of claim 18, wherein the topical dermatological composition is concurrently administered with at least one of an oral antibiotic, oral anti-androgen, and oral retinoid.

24. The method of claim 18, where the topical dermatological composition comprises diclofenac and clindamycin that are both independently microencapsulated.

25. The method of claim 18, where the topical dermatological composition further comprises at least one of:
(i) antibiotic;
(ii) antibacterial agent;
(iii) retinoid;
(iv) anti-androgenic agent;
(v) folate;
(vi) keratolytic agent;
(vii) at least one of azelaic acid, alpha hydroxy acid, trichloroacetic acid, lactic acid, ethanol, resorcinol, sulfur, sodium sulfacetamide, ivermectin, and glycolic acid; and
(viii) penetrating agent.

26. The method of claim 18, where the topical dermatological composition comprises diclofenac that is microencapsulated in a liposome.

27. The method of claim 18, where the topical dermatological composition comprises clindamycin that is microencapsulated in a liposome.

28. The method of claim 18, where the topical dermatological composition comprises diclofenac present in up to about 10 wt. % of the topical dermatological composition.

29. The method of claim 18, where the topical dermatological composition comprises clindamycin present in up to about 5 wt. % of the topical dermatological composition.

30. A method of treating acne vulgaris in a human patient, the method comprising topically administering to a human patient in need thereof, a topical dermatological composition comprising, in a pharmaceutically acceptable medium:
diclofenac, present in up to about 10 wt. % of the topical dermatological composition; and
clindamycin, present in up to about 5 wt. % of the topical dermatological composition;
the topical dermatological composition further comprising at least one of (i)-(viii):
(i) antibiotic;
(ii) antibacterial agent;
(iii) retinoid;
(iv) anti-androgenic agent;
(v) folate;
(vi) keratolytic agent;
(vii) at least one of azelaic acid, alpha hydroxy acid, trichloroacetic acid, lactic acid, ethanol, resorcinol, sulfur, sodium sulfacetamide, ivermectin, and glycolic acid; and
(viii) penetrating agent;
wherein at least one of the diclofenac and clindamycin is at least partially microencapsulated in the form of liposome;
wherein the dermatological composition is administered in an amount and for a period of time effective to treat the acne vulgaris.
